(19) United States
(12) Patent Application Publication
(10) Pub. No.: US 2025/0283036 A1
(43) Pub. Date: Sep. 11, 2025

(54) ENGINEERING B CELL-BASED PROTEIN FACTORIES TO TREAT SERIOUS DISEASES

(71) Applicant: Walking Fish Therapeutics, Inc., South San Francisco, CA (US)

(72) Inventors: Rosa ROMANO, San Mateo, CA (US); Hangil PARK, San Francisco, CA (US); Weijie LAN, Belmont, CA (US)

(21) Appl. No.: 18/556,540

(22) PCT Filed: Apr. 20, 2022
(86) PCT No.: PCT/US2022/025471

§ 371 Date: Oct. 20, 2023

Related U.S. Application Data
(60) Provisional application No. 63/176,944, filed on Apr. 20, 2021.

(51) Int. Cl. C12N 5/0781; A61K 35/17; A61K 38/37; A61K 38/44; A61K 38/47; A61K 38/51; C12N 9/22; C12N 15/11

(52) U.S. Cl. CPC C12N 5/0635; A61K 35/17; A61K 38/37; A61K 38/44; A61K 38/47; A61K 38/51; C12N 9/226; C12N 15/111; C12N 2310/20; C12N 2510/00

(57) ABSTRACT

The invention(s) disclosed herein relate to improved methods for expanding cell populations, particularly B cell populations. The invention further relates comprising improved cell media, compositions thereof, and methods of using such expanded B cells. Wherein a population of cells comprises engineered human B cells, wherein the engineered human B cells comprise a therapeutic protein, whose gene has been inserted into the beta-2M locus.

### CROSS-REFERENCE TO RELATED APPLICATIONS

This patent application is a national stage application of International Application No. PCT/US2022/025471 entitled “ENGINEERING B CELL-BASED PROTEIN FACTORIES TO TREAT SERIOUS DISEASES,” filed on Apr. 20, 2022, which claims the benefit of U.S. Provisional Patent Application Ser. No. 63/176,944, filed on Apr. 20, 2021, each of which are incorporated by reference herein in their entirety for all purposes.

### SEQUENCE LISTING

The instant application contains a Sequence Listing which has been submitted electronically in ASCII format and is hereby incorporated by reference in its entirety. Said ASCII copy, created on Jan. 31, 2024, is named 2219235_00121US2_SL.txt and is 144,951 bytes in size.

### BACKGROUND OF THE INVENTION

B cells are naturally hardwired to present antigens and secrete immunoglobulins. Theoretically, B cells should have great potential as a cellular therapy for targeting certain diseased cell types and expressing therapeutic proteins. There thus exists a need for alternative treatments, such as genetically engineered B cells, for the treatment of a variety of diseases and disorders, including cancer, heart disease, inflammatory disease, muscle wasting disease, neurological disease, and the like. Modifying B cells for the treatment of various diseases, however, is a technique that has not been extensively studied, despite the critical role of B cells in immune responses. Human B cells are easily isolated and can be expanded, making them viable candidates for engineering. Importantly, B cells can be matured to long-lived cells that are ideal for provision of therapeutic proteins that are required for extended periods. Indeed, injected B cells can traffic to their normal tissue niches including spleen, lymph nodes and the bone marrow where they can persist. Ex vivo modification is desirable to avoid in vivo use of recombinant viruses to which immune responses can inactivate. Collectively, B cells are ideal as therapeutic delivery vehicles.

### SUMMARY OF THE INVENTION

In various embodiments, the invention disclosed herein relates to genetically engineering B cells to express a therapeutic protein.

In various embodiments, the invention relates to a population of cells comprising engineered human B cells, wherein the engineered human B cells comprise a therapeutic protein, whose gene has been inserted into the β2M locus.

In various embodiments, the engineered human B cells further comprise a disrupted β2M gene. In various embodiments, the nucleic acid sequence capable of expressing the therapeutic payload has been inserted into exon 2 of the β2M locus. In various embodiments, the nucleic acid sequence capable of expressing the therapeutic payload has been inserted into an intron of the β2M locus, such that β2M expression is maintained at a percentage of greater than 50%. In various embodiments, the therapeutic protein is alpha-galactosidase A (GLA), acid alpha-glucosidase (GAA), phenylalanine hydroxylase (PAH), phenylalanine ammonia-lyase (PAL) or full length or B domain deleted (BDD) FVIII. In various embodiments, the therapeutic protein is selected from the amino acid sequences consisting of SEQ ID NOs. 2-6. In various embodiments, the therapeutic protein is a GPC3 chimeric receptor. In various embodiments, the GPC3 chimeric receptor comprises an amino acid sequence of SEQ ID NO. 16. In various embodiments, the therapeutic protein is a cytokine or a chemokine. In various embodiments, the cytokine is IL-10. In various embodiments, the cytokine comprises an amino acid sequence of SEQ ID NO. 7. In various embodiments, the expression of the endogenous β2M has been reduced by at least 40%. In various embodiments, the endogenous β2M has been reduced by at least 80%. In various embodiments, the population of cells, express said therapeutic proteins.

In various embodiments, the invention relates to a population of cells comprising engineered human B cells, wherein the engineered human B cells comprise, a disrupted β2M gene; and a therapeutic protein, whose gene has been inserted into the β2M locus, wherein the therapeutic protein selected from the amino acid sequences consisting of SEQ ID NOs. 2-7, wherein a nucleic acid sequence capable of expressing the therapeutic payload has been inserted into exon 2 of the β2M locus, wherein expression of the endogenous β2M has been reduced by at least 40%; and wherein at least 20% of the population of cells, express said therapeutic proteins.

In various embodiments, the invention relates to a method of producing an engineered B cell expressing a therapeutic protein, the method comprising delivering to a human B cell, an RNA-guided nuclease, a gRNA targeting the β2M gene, a construct comprising a nucleic acid sequence encoding a therapeutic protein.

In various embodiments, the RNA-guided nuclease and gRNA targeting the β2M gene are delivered to the B cell as an RNP. In various embodiments, the RNA-guided nuclease and gRNA targeting the β2M gene are delivered to the B cell as a nanoparticle. In various embodiments, the RNA-guided nuclease and gRNA targeting the β2M gene are delivered to the B cell via electroporation. In various embodiments, the construct delivered to the B cell using a viral vector. In various embodiments, the construct delivered to the B cell as DNA. In various embodiments, the RNA-guided nuclease comprises the nucleotide sequence of SEQ ID NO. 18. In various embodiments, the gRNA comprises the nucleic acid sequence of SEQ ID NO. 19. In various embodiments, the gRNA specifically targets exon 2 of the B2M locus. In various embodiments, the gRNA specifically targets an intron of the B2M locus. In various embodiments, the β2M expression is maintained at a percentage of greater than 50%. In various embodiments, the targeting construct comprises a codon-optimized nucleic acid sequence selected from the group consisting of SEQ ID NOs. 10-17 and 31. In various embodiments, the construct comprises a left homology arm of SEQ ID NO. 20 and a right homology arm of SEQ ID NO. 21. In various embodiments, the expression of the endogenous β2M has been reduced by at least 40%. In various embodiments, the expression of the endogenous β2M has been reduced by at least 80%. In various embodiments, the at least 20% of the engineered B cells, express said therapeutic protein.

In various embodiments, the invention relates to a method of producing an engineered B cell expressing a therapeutic protein, the method comprising delivering to a human B cell, comprising a RNA-guided nuclease, wherein the RNA-guided nuclease comprises the amino acid sequence of SEQ ID NO. 18; a gRNA targeting the B2M gene, wherein the gRNA comprises the nucleic acid sequence of SEQ ID NO. 19; a construct comprising a nucleic acid sequence encoding a therapeutic protein; wherein the construct comprises a nucleic acid sequence selected from the group consisting of SEQ ID NOs. 10-17 and 31; wherein the construct further comprises a left homology arm of SEQ ID NO. 22 and a right homology arm of SEQ ID NO. 21, wherein expression of the endogenous B2M has been reduced by at least 40%; and wherein at least 20% of the engineered B cells, express said therapeutic protein.

In various embodiments, the invention relates to a method of treating a patient in need thereof, by administering to said patient a population of cells comprising engineered human B cells, wherein the engineered human B cells comprise a therapeutic payload, whose gene has been inserted into the β2M locus.

In various embodiments, the engineered human B cells further comprise a disrupted β2M gene. In various embodiments, the nucleic acid sequence capable of expressing the therapeutic payload has been inserted into exon 2 of the β2M locus. In various embodiments, the nucleic acid sequence capable of expressing the therapeutic payload has been inserted into an intron of the β2M locus, such that β2M expression is maintained at a percentage of greater than 50%. In various embodiments, the nucleic acid sequence capable of expressing the therapeutic payload has been inserted into exon 2 of the β2M locus. In various embodiments, the RNA-guided nuclease and gRNA targeting the β2M gene are delivered to the B cell as an RNP. In various embodiments, the RNA-guided nuclease and gRNA targeting the β2M gene are delivered to the B cell as a nanoparticle. In various embodiments, the RNA-guided nuclease and gRNA targeting the β2M gene are delivered to the B cell via electroporation. In various embodiments, the construct delivered to the B cell using a viral vector. In various embodiments, the construct delivered to the B cell as DNA. In various embodiments, the therapeutic protein is alpha-galactosidase A (GLA), acid alpha-glucosidase (GAA), phenylalanine hydroxylase (PAH), phenylalanine ammonia-lyase (PAL) or B domain deleted (BDD) FVIII. In various embodiments, the therapeutic protein selected from the amino acid sequences consisting of SEQ ID NOs. 2-7. In various embodiments, the therapeutic protein is a GPC3 chimeric receptor. In various embodiments, the GPC3 chimeric receptor comprises an amino acid sequence of SEQ ID NO. 16. In various embodiments, the therapeutic protein is a cytokine or a chemokine. In various embodiments, the cytokine is IL-10. In various embodiments, the cytokine is SEQ ID NO. 9. In various embodiments, the expression of the endogenous β2M has been reduced by at least 40%. In various embodiments, the expression of the endogenous β2M has been reduced by at least 80%. In various embodiments, at least 20% of the population of cells, express said therapeutic protein. In various embodiments, the disease or disorder is Fabry disease, Pompe disease, Phenylketonuria (PKU) or Hemophilia A.

In various embodiments, the invention relates to method of treating a patient in need thereof comprising administering to said patient a population of cells comprising engineered human B cells, wherein the engineered human B cells comprise, a disrupted β2M gene; and a therapeutic payload, whose gene has been inserted into the β2M locus, wherein the therapeutic protein selected from the amino acid sequences consisting of SEQ ID NOs. 2-7; wherein a nucleic acid sequence capable of expressing the therapeutic payload has been inserted into exon 2 of the β2M locus; wherein expression of the endogenous β2M has been reduced by at least 40%; and wherein at least 20% of the population of cells, express said therapeutic proteins.

In various embodiments, the invention relates to a genome editing system, comprising an RNA-guided nuclease; a gRNA targeting the β2M gene; and a construct comprising a nucleic acid sequence encoding a therapeutic protein.

In various embodiments, the RNA-guided nuclease comprises the amino acid sequence of SEQ ID NO. 18. In various embodiments, the gRNA comprises the nucleic acid sequence of SEQ ID NO. 19. In various embodiments, the construct comprises a nucleic acid sequence selected from the group consisting of SEQ ID NOs. 10-17 and 31. In various embodiments, the construct comprises a left homology arm of SEQ ID NO. 21 and a right homology arm of SEQ ID NO. 22. In various embodiments, the expression of the endogenous β2M has been reduced by at least 40%. In various embodiments, the expression of the endogenous β2M has been reduced by at least 80%. In various embodiments, at least 20% of the engineered B cells, express said therapeutic protein.

In various embodiments, the invention relates to a genome editing system, comprising, a RNA-guided nuclease, wherein the RNA-guided nuclease comprises the amino acid sequence of SEQ ID NO. 8; a gRNA targeting the β2M gene, wherein the gRNA comprises the nucleic acid sequence of SEQ ID NO. 9; and a construct comprising a nucleic acid sequence encoding a therapeutic protein; wherein the construct comprises a nucleic acid sequence selected from the group consisting of SEQ ID NOs. 10-16; wherein the construct further comprises a left homology arm of SEQ ID NO. 17 and a right homology arm of SEQ ID NO. 18; wherein expression of the endogenous β2M has been reduced by at least 40%; and wherein at least 20% of the engineered B cells, express said therapeutic protein.

In various embodiments, the invention relates to an engineered B cell comprising a nucleic acid sequence capable of expressing an amino acid sequence comprising SEQ ID NO. 2. In various embodiments, the invention relates to an engineered B cell comprising a nucleic acid sequence capable of expressing an amino acid sequence comprising SEQ ID NO. 3. In various embodiments, the invention relates to an engineered B cell comprising a nucleic acid sequence capable of expressing an amino acid sequence comprising SEQ ID NO. 4. In various embodiments, the invention relates to an engineered B cell comprising a nucleic acid sequence capable of expressing an amino acid sequence comprising SEQ ID NO. 5. In various embodiments, the invention relates to an engineered B cell comprising a nucleic acid sequence capable of expressing an amino acid sequence comprising SEQ ID NO. 6. In various embodiments, the invention relates to an engineered B cell comprising a nucleic acid sequence capable of expressing an amino acid sequence comprising SEQ ID NO. 7.

In various embodiments, the invention relates to a nucleic acid construct capable of insertion into the β2M locus of a B cell, comprising the nucleic acid sequence of SEQ ID NO. 10. In various embodiments, the invention relates to a nucleic acid construct capable of insertion into the β2M locus of a B cell, comprising the nucleic acid sequence of SEQ ID NO. 11. In various embodiments, the invention relates to a nucleic acid construct capable of insertion into the β2M locus of a B cell, comprising the nucleic acid sequence of SEQ ID NO. 12. In various embodiments, the invention relates to a nucleic acid construct capable of insertion into the β2M locus of a B cell, comprising the nucleic acid sequence of SEQ ID NO. 13. In various embodiments, the invention relates to a nucleic acid construct capable of insertion into the β2M locus of a B cell, comprising the nucleic acid sequence of SEQ ID NO. 14. In various embodiments, the invention relates to a nucleic acid construct capable of insertion into the β2M locus of a B cell, comprising the nucleic acid sequence of SEQ ID NO. 15. In various embodiments, the invention relates to a nucleic acid construct capable of insertion into the β2M locus of a B cell, comprising the nucleic acid sequence of SEQ ID NO. 16. In various embodiments, the invention relates to a nucleic acid construct capable of insertion into the β2M locus of a B cell, comprising the nucleic acid sequence of SEQ ID NO. 17. In various embodiments, the invention relates to a nucleic acid construct capable of insertion into the β2M locus of a B cell, comprising the nucleic acid sequence of SEQ ID NO. 31.

### BRIEF DESCRIPTION OF THE FIGURES

FIG. 1 shows a schematic of human B cell preparation for CRISPR engineering: isolation, activation and expansion. PBMCs were isolated from buffy coats using Ficoll-Paque. Primary human B cells were isolated using the EASYSEP™ Human B Cell Isolation Kit. Isolated B cells were activated and expanded using the human B Cell Expansion Kit for over 9 days. Harvested B cells were engineered with nucleofection using AMAXA™ 4D-NUCLEOFACTOR™. Engineered B cells were cultured and analyzed by PCR and flow cytometry.

FIGS. 2A-2B show optimal human B cell nucleofection protocol development. FIG. 2A shows the screen of indicated electroporation programs for optimal human B cell nucleofection using Amaxa 4D. 1 μg pMAX-GFP was used for each condition with 1 million activated human B cells in buffer P3. Nuclefection efficiency was determined by GFP expression using flow cytometry. FIG. 2B shows a table summary of nucleofection efficiency and cell viability for each electroporation program. Program CM-137 was selected as the optimal electroporation program.

FIG. 3 shows a schematic of the design of the CRISPR guide RNA for engineering of human β2M locus. CRISPR guide sequence CGTGAGTAAACCTGAATCTT (SEQ ID NO: 34) was selected to target the beginning of exon 2 of the human β2M gene to efficiently knock-out express of functional β2M protein. Phosphorothioated 2′ O-Methyl modified single guide RNA (sgRNA) oligomer was synthesized by IDT to improve CRISPR efficiency. Figure discloses SEQ ID No: 24.

FIGS. 4A-4C show optimal human B cell CRISPR editing protocol development. FIG. 4A shows a schematic of RNP formation for CRISPR gene editing. Cas9 enzyme was incubated with sgRNA at a 1:1.2 ratio for 10 minutes at room temperature to form RNP. 100 pmol RNA was used for 1 million B cells in a 20 μL reaction. FIG. 4B shows flow profiles of control and β2M CRISPR edited B cells. Successful β2M CRISPR editing resulted in loss of β2M expression on the cell surface, which was determined by flow cytometry. Overlay of the profiles illustrated efficient knock-out of β2M. FIG. 4C shows a table summary of β2M knock-out efficiency and cell viability for each electroporation program. Program CM-137 was selected as the optimal β2M knock-out program.

FIGS. 5A-5B show validation of β2M KO frequency at the genomic level. FIG. 5A shows human B cells were isolated from PBMCs (1 healthy donor) and electroporated with WT-Cas9 complex with β2M sgRNA. Two days after targeting, genomic DNA was extracted Sanger sequencing was used to quantify INDELs. FIG. 5B shows an overview of the insertions and deletions generated at the cut site of the β2M sgRNA. Analysis was performed using ICE synthego (ice.synthego.com) web-based software. (SEQ ID NOs: 34-50, respectively, in order of appearance.)

FIG. 6 shows a schematic of the design of promoter-less β2M targeting constructs, based on the following principles: 1) only correctly targeted alleles express the transgene (GFP or GPC3-CAR), 2) high level constitutive expression of transgene driven from endogenous β2M promoter, and 3) loss of β2M expression on engineered B cells allows for easy detection of successful editing.

FIG. 7 shows a schematic of β2M gene pre- and post-editing where GFP expression is driven from the endogenous β2M promoter.

FIGS. 8A-8C show human B-cell expansion and β2M editing protocol. FIG. 8A shows a schematic of the B cells editing procedure. Human B cells were isolated from healthy donor PBMCs and expanded in presence of CD40L and IL-4 for 9 days. Expanded cells were electroporated with RNP (WT-Cas9 and β2M sgRNA) and transduced with AAV6 to deliver GFP and GPC3-CAR HDR-donor cassettes. FIG. 8B shows a growth curve of cultured human B cells. FIG. 8C shows the viability of cultured human B cells over the course of expansion.

FIGS. 9A-9D show the efficient AAV6-mediated integration at the β2M locus in activated human B cells. To determine the rates of targeted integration of the GFP and the GPC3-CAR promoter-less HDR-donors, expression of GFP (FIG. 9A) and GPC3-CAR (FIG. 9B) in engineered B cells was evaluated using flow cytometry at 3 or 6 days after editing. Using an MOI of 100K, AAV6 mediated targeting efficiency was >40% for GFP or aGPC3-CAR at β2M locus. Viability of GFP (FIG. 9C) and GPC3-CAR (FIG. 9D) engineered B cells was measured using Trypan Blue at 3 or 6 days after editing.

FIGS. 10A-10B show the results of flow cytometry analysis after using PCR dsDNA as an HDR template for CRISPR-mediated targeting of human B cells at β2M locus. GFP dsDNA PCR product was used as an HDR template for CRISPR engineering of human B cells in combination with Cas9 RNP. Three μg of donor DNA resulted in 15% targeting efficiency as determined by flow cytometry analysis of GFP expression. Flow cytometry analysis is shown without (FIG. 10A) and with RNP electroporation (FIG. 10B).

FIG. 11 shows schematic of the design of CRISPR-mediated β2M locus targeting for the stable expression of enzymes for replacement therapy.

FIG. 12 shows a schematic of the design of CRISPR-mediated β2M locus targeting for the stable expression of cytokines as payload.

FIG. 13 shows a schematic of the design of CRISPR-mediated β2M locus targeting for the stable expression of wild type GLA as payload.

FIG. 14 shows secreted (FIG. 14A) and intracellular (FIG. 14B) GLA expression in B cells engineered using Cas9-rAAV to express wild type GLA. The targeting loci was exon 2 of the β2M locus and GLA expression was driven by the endogenous β2M promoter.

### DETAILED DESCRIPTION

The present disclosure provides an efficient gene method for transgene integration into the β2M locus for cell therapy. The present disclosure is based, at least in part, on the discovery that insertion of a therapeutic protein into the β2M locus in B cells using gene editing technologies enhances several characteristics important for cell-based immunotherapy. For example, targeted expression of a therapeutic protein from the β2M locus takes advantage of the high basal level of β2M expression in B cells to achieve a high level and ubiquitous expression of the transgene/therapeutic protein across different B cell types independent of developmental stage and activation status.

Disclosed herein are a number of constructs for insertion into the β2M locus. The invention disclosed herein would be suitable for any number of therapies that require delivery or replacement of a therapeutic protein such as a therapeutic enzyme, an antibody, a cytokine a selection marker, a suicide gene, etc.

In certain embodiments, the optimized gene editing methods deliver the transgene, which is inserted into an exon of the β2M gene. Such methods are capable of achieving a greater than 50% knockout of the endogenous β2M gene in human B cells. In other embodiments of the present disclosure, the transgene is inserted using gene editing methods into an intron of the β2M gene, such that β2M gene expression is not disrupted or is only minimally disrupted.

The present disclose is capable of achieving over 50% targeted integration efficiency.

### I. Definitions

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. As utilized in accordance with the present disclosure, the following terms, unless otherwise indicated, shall be understood to have the following meanings:

In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The term “polynucleotide”, “nucleotide”, or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′, 3′-dideoxyribose, and internucleotide linkage modifications such as phosphorothioate, phosphorodithioate, phosphoroselenoate, phosphoro-diselenoate, phosphoro-anilothioate, phoshoraniladate and phosphoroamidate.

The term “oligonucleotide” refers to a polynucleotide comprising 200 or fewer nucleotides. Oligonucleotides can be single stranded or double stranded, e.g., for use in the construction of a mutant gene. Oligonucleotides can be sense or antisense oligonucleotides. An oligonucleotide can include a label, including a radiolabel, a fluorescent label, a hapten or an antigenic label, for detection assays. Oligonucleotides can be used, for example, as PCR primers, cloning primers or hybridization probes.

The term “control sequence” refers to a polynucleotide sequence that can affect the expression and processing of coding sequences to which it is ligated. The nature of such control sequences can depend upon the host organism. In particular embodiments, control sequences for prokaryotes can include a promoter, a ribosomal binding site, and a transcription termination sequence. For example, control sequences for eukaryotes can include promoters comprising one or a plurality of recognition sites for transcription factors, transcription enhancer sequences, and transcription termination sequence. Control sequences can include leader sequences (signal peptides) and/or fusion partner sequences.

As used herein, “operably linked” means that the components to which the term is applied are in a relationship that allows them to carry out their inherent functions under suitable conditions.

The term “vector” means any molecule or entity (e.g., nucleic acid, plasmid, bacteriophage or virus) used to transfer protein coding information into a host cell. The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct can include, but is not limited to, sequences that affect or control transcription, translation, and, if introns are present, affect RNA splicing of a coding region operably linked thereto.

The term “host cell” refers to a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell, or can be maintained transiently as an episomal element without being replicated, or can replicate independently as a plasmid. A cell is considered to have been “stably transformed” when the transforming DNA is replicated with the division of the cell.

The term “transfection” refers to the uptake of foreign or exogenous DNA by a cell. A number of transfection techniques are well known in the art and are disclosed herein. See, e.g., Graham et al., 1973, Virology, 1973, 52:456; Sambrook et al., Molecular Cloning: A Laboratory Manual, 2001, supra; Davis et al., Basic Methods in Molecular Biology, 1986, Elsevier; Chu et al., 1981, Gene, 13:197.

The term “transduction” refers to the process whereby foreign DNA is introduced into a cell via viral vector. See, e.g., Jones et al., Genetics: Principles and Analysis, 1998, Boston: Jones & Bartlett Publ.

The terms “polypeptide” or “protein” refer to a macromolecule having the amino acid sequence of a protein, including deletions from, additions to, and/or substitutions of one or more amino acids of the native sequence. The terms “polypeptide” and “protein” specifically encompass antigen-binding molecules, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. Useful polypeptide fragments include immunologically functional fragments of antigen-binding molecules.

The term “isolated” means (i) free of at least some other proteins with which it would normally be found, (ii) is essentially free of other proteins from the same source, e.g., from the same species, (iii) separated from at least about 50 percent of polynucleotides, lipids, carbohydrates, or other materials with which it is associated in nature, (iv) operably associated (by covalent or noncovalent interaction) with a polypeptide with which it is not associated in nature, or (v) does not occur in nature.

A “variant” of a polypeptide (e.g., an antigen-binding molecule) comprises an amino acid sequence wherein one or more amino acid residues are inserted into, deleted from and/or substituted into the amino acid sequence relative to another polypeptide sequence. Variants include fusion proteins.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”).

To calculate percent identity, the sequences being compared are typically aligned in a way that gives the largest match between the sequences. One example of a computer program that can be used to determine percent identity is the GCG program package, which includes GAP (Devereux et al., Nucl. Acid Res., 1984, 12, 387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). In certain embodiments, a standard comparison matrix (see, e.g., Dayhoff et al., 1978, Atlas of Protein Sequence and Structure, 5:345-352 for the PAM 250 comparison matrix; Henikoff et al., 1992, Proc. Natl. Acad. Sci. U.S.A., 89, 10915-10919 for the BLO-SUM 62 comparison matrix) is also used by the algorithm.

As used herein, the twenty conventional (e.g., naturally occurring) amino acids and their abbreviations follow conventional usage. See, e.g., Immunology A Synthesis (2nd Edition, Golub and Green, Eds., Sinauer Assoc., Sunderland, Mass. (1991)), which is incorporated herein by reference for any purpose. Stereoisomers (e.g., D-amino acids) of the twenty conventional amino acids, unnatural amino acids such as alpha-, alpha-disubstituted amino acids, N-alkyl amino acids, lactic acid, and other unconventional amino acids can also be suitable components for polypeptides of the present invention. Examples of unconventional amino acids include: 4-hydroxyproline, gamma.-carboxy-glutamate, epsilon-N,N,N-trimethyllysine, e-N-acetyllysine, 0-phosphoserine, N-acetylserine, N-formylmethionine, 3-methylhistidine, 5-hydroxylysine, sigma.-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Conservative amino acid substitutions can encompass non-naturally occurring amino acid residues, which are typically incorporated by chemical peptide synthesis rather than by synthesis in biological systems. These include peptidomimetics and other reversed or inverted forms of amino acid moieties. Naturally occurring residues can be divided into classes based on common side chain properties:
a) hydrophobic: norleucine, Met, Ala, Val, Leu, Ile;
b) neutral hydrophilic: Cys, Ser, Thr, Asn, Gln;
c) acidic: Asp, Glu;
d) basic: His, Lys, Arg;
e) residues that influence chain orientation: Gly, Pro; and
f) aromatic: Trp, Tyr, Phe.

For example, non-conservative substitutions can involve the exchange of a member of one of these classes for a member from another class.

In making changes to the antigen-binding molecule, the costimulatory or activating domains of the engineered T cell, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). See, e.g., Kyte et al., 1982, J. Mol. Biol., 157, 105-131. It is known that certain amino acids can be substituted for other amino acids having a similar hydropathic index or score and still retain a similar biological activity. It is also understood in the art that the substitution of like amino acids can be made effectively on the basis of hydrophilicity, particularly where the biologically functional protein or peptide thereby created is intended for use in immunological embodiments, as in the present case. Exemplary amino acid substitutions are set forth in Table 1.

TABLE 1

| Original | Exemplary | Preferred |
|---|---|---|
| Residues | Substitutions | Substitutions |
| Ala | Val, Leu, Ile | Val |
| Arg | Lys, Gin, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Va, Met, Ala, Phe | Ile |
| Lys | Arg, 1,4 Diamino-butyric | Arg |
| | Acid, Gin, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen-binding molecule can have a greater circulating half-life than an antigen-binding molecule that is not chemically modified. In some embodiments, a derivative antigen-binding molecule is covalently modified to include one or more water-soluble polymer attachments, including, but not limited to, polyethylene glycol, polyoxyethylene glycol, or polypropylene glycol.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. L., 1986, Adv. Drug Res., 1986, 15, 29; Veber, D. F. & Freidinger, R. M., 1985, Trends in Neuroscience, 8, 392-396; and Evans, B. E., et al., 1987, J Med. Chem., 30, 1229-1239, which are incorporated herein by reference for any purpose.

The term “therapeutically effective amount” refers to the amount of immune cells or other therapeutic agent determined to produce a therapeutic response in a mammal. Such therapeutically effective amounts are readily ascertained by one of ordinary skill in the art.

The terms “patient” and “subject” are used interchangeably and include human and non-human animal subjects as well as those with formally diagnosed disorders, those without formally recognized disorders, those receiving medical attention, those at risk of developing the disorders, etc.

The term “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. The term “prevent” does not require the 100% elimination of the possibility of an event. Rather, it denotes that the likelihood of the occurrence of the event has been reduced in the presence of the compound or method.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% higher compared to a reference quantity, level, value, number, frequency, percentage, dimension, size, amount, weight or length. In one embodiment, the terms “essentially the same” or “substantially the same” refer to a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance, such as, 95% free, 96% free, 97% free, 98% free, 99% free of the specified substance, or is undetectable as measured by conventional means. Similar meaning can be applied to the term “absence of,” where referring to the absence of a particular substance or component of a composition.

As used herein, the term “appreciable” refers to a range of quantity, level, value, number, frequency, percentage, dimension, size, amount, weight or length or an event that is readily detectable by one or more standard methods. The terms “not-appreciable” and “not appreciable” and equivalents refer to a range of quantity, level, value, number, frequency, percentage, dimension, size, amount, weight or length or an event that is not readily detectable or undetectable by standard methods. In one embodiment, an event is not appreciable if it occurs less than 5%, 4%, 3%, 2%, 1%, 0.1%, 0.001%, or less of the time.

Throughout this specification, unless the context requires otherwise, the words “comprise,” “comprises” and “comprising” will be understood to imply the inclusion of stated step or element or group of steps or elements but not the exclusion of any other step or element or group of steps or elements. In particular embodiments, the terms “include,” “has,” “contains,” and “comprise” are used synonymously.

As used herein, “consisting of” is meant including, and limited to, whatever follows the phrase “consisting of”. Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” or “approximately” refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5% or 1%, or any intervening ranges thereof.

As used herein, the term “introducing” refers to a process that comprises contacting a cell with a polynucleotide, polypeptide, or small molecule. An introducing step may also comprise microinjection of polynucleotides or polypeptides into the cell, use of liposomes to deliver polynucleotides or polypeptides into the cell, or fusion of polynucleotides or polypeptides to cell permeable moieties to introduce them into a cell.

### II. Gene Editing Methods

Gene editing (including genomic editing) is a type of genetic engineering in which nucleotide(s)/nucleic acid(s) is/are inserted, deleted, and/or substituted in a DNA sequence, such as in the genome of a targeted cell. Targeted gene editing enables insertion, deletion and/or substitution at pre-selected sites in the genome of a targeted cell (e.g., in a targeted gene or targeted DNA sequence). When a sequence of an endogenous gene is edited, for example by deletion, insertion or substitution of nucleotide(s)/nucleic acid(s), the endogenous gene comprising the affected sequence may be knocked-out or knocked-down due to the sequence alteration. Therefore, targeted editing may be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present. As used herein, a “disrupted gene” refers to a gene comprising an insertion, deletion, or substitution relative to an endogenous gene such that expression of a functional protein from the endogenous gene is reduced or inhibited. As used herein, “disrupting a gene” refers to a method of inserting, deleting or substituting at least one nucleotide/nucleic acid in an endogenous gene such that expression of a functional protein from the endogenous gene is reduced or inhibited. Methods of disrupting a gene are known to those of skill in the art and described herein.

Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide may introduce deletions, insertions or replacement of nucleotides in the endogenous sequence.

Alternatively, the nuclease-dependent approach can achieve targeted editing with higher frequency through the specific introduction of double strand breaks (DSBs) by specific rare-cutting nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

Available endonucleases capable of introducing specific and targeted DSBs include, but are not limited to, zinc-finger nucleases (ZFN), meganucleases, transcription activator-like effector nucleases (TALEN), and RNA-guided CRISPR Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases may also be used for targeted integration.

ZFNs are targeted nucleases comprising a nuclease fused to a zinc finger DNA binding domain (ZFBD), which is a polypeptide domain that binds DNA in a sequence specific manner through on more zinc fingers. A zinc finger is a domain of about 30 amino acids within the zinc finger-binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the Fok1 nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable diresidues (RVD). TALENs are described in greater detail in US Patent Application No. 2011/0145940. The most recognized example of a TALEN in the art is a fusion polypeptide of the Fok1 nuclease to a TAL effector DNA binding domain.

Additional examples of targeted nucleases suitable for use as provided herein include, but are not limited to, Bxb1, phiC31, R4, PhiBT1, and WB/SPBc/TP901-1, whether used individually or in combination.

Other non-limiting examples of targeted nucleases include naturally-occurring and recombinant nucleases, e.g., CRISPR/Cas9, restriction endonucleases, meganucleases homing endonucleases, and the like.

### 1. CRISPR-Cas9 Gene Editing

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (CrRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon reintroduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, single-guide RNA (sgRNA), if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end-joining (NHEJ) and homology-directed repair (HDR).

NHEJ is a robust repair mechanism that appears highly active in the majority of cell types, including nondividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.

In some embodiments, the Cas9 (CRISPR associated protein 9) endonuclease is from Streptococcus pyogenes, although other Cas9 homologs may be used. It should be understood, that wild-type Cas9 may be used or modified versions of Cas9 may be used (e.g., evolved versions of Cas9, or Cas9 orthologues or variants), as provided herein. In some embodiments, Cas9 may be substituted with another RNA-guided endonuclease, such as Cpf1 (of a class II CRISPR/Cas system).

In some embodiments, the CRISPR/Cas system comprise components derived from a Type-1, Type-II, or Type-III system. Updated classification schemes for CRISPR/Cas loci define Class 1 and Class 2 CRISPR/Cas systems, having Types I to V or VI (Makarova et al., (2015) Nat Rev Microbiol, 13(11):722-36; Shmakov et al., (2015)) Mol Cell, 60:385-397). Class 2 CRISPR/Cas systems have single protein effectors. Cas proteins of Types II, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163: 1-13) is homologous to Cas9, and contains a RuvC-like nuclease domain.

In some embodiments, the Cas nuclease is from a Type-II CRISPR/Cas system (e.g., a Cas9 protein from a CRISPR/Cas9 system). In some embodiments, the Cas nuclease is from a Class 2 CRISPR Cas system (a single protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

In some embodiments, a Cas nuclease may comprise more than one nuclease domain. For example, a Cas9 nuclease may comprise at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease).

In some embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

### 2. Guide RNAs

The present disclosure provides a genome-targeting nucleic acid that can direct the activities of an associated polypeptide (e.g., a site-directed polypeptide) to a specific target sequence within a target nucleic acid. The genome-targeting nucleic acid can be an RNA. A genome-targeting RNA is referred to as a “guide RNA” or “gRNA” herein. A guide RNA comprises at least a spacer sequence that hybridizes to a target nucleic acid sequence of interest, and a CRISPR repeat sequence. In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins.

A single-molecule guide RNA in a Type V system comprises, in the 5′ to 3′ direction, a minimum CRISPR repeat sequence and a spacer sequence.

In some embodiments, the sgRNA comprises a 20 nucleotide spacer sequence at the 5′ end of the sgRNA sequence. In some embodiments, the sgRNA comprises a less than 20 nucleotide spacer sequence at the 5′ end of the sgRNA sequence. In some embodiments, the sgRNA comprises a more than 20 nucleotide spacer sequence at the 5′ end of the sgRNA sequence.

In some embodiments, the sgRNA comprises comprise no uracil at the 3′ end of the sgRNA sequence. In some embodiments, the sgRNA comprises comprise one or more uracil at the 3′ end of the sgRNA sequence. For example, the sgRNA can comprise 1 uracil (U) at the 3′ end of the sgRNA sequence. The sgRNA can comprise 2 uracil (UU) at the 3′ end of the sgRNA sequence. The sgRNA can comprise 3 uracil (UUU) at the 3′ end of the sgRNA sequence. The sgRNA can comprise 4 uracil (UUUU) at the 3′ end of the sgRNA sequence. The sgRNA can comprise 5 uracil (UUUUU) at the 3′ end of the sgRNA sequence. The sgRNA can comprise 6 uracil (UUUUUU) at the 3′ end of the sgRNA sequence. The sgRNA can comprise 7 uracil (UUUUUUU) at the 3′ end of the sgRNA sequence. The sgRNA can comprise 8 uracil (UUUUUUUU) at the 3′ end of the sgRNA sequence.

The sgRNA can be unmodified or modified. For example, modified sgRNAs can comprise one or more 2′-O-methyl phosphorothioate nucleotides.

By way of illustration, guide RNAs used in the CRISPR/Cas/Cpf1 system, or other smaller RNAs can be readily synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some embodiments, indel frequency (editing frequency) may be determined using a TIDE analysis, which can be used to identify highly efficient gRNA molecules. In some embodiments, a highly efficient gRNA yields a gene editing frequency of higher than 80%. For example, a gRNA is considered to be highly efficient if it yields a gene editing frequency of at least 80%, at least 85%, at least 90%, at least 95%, or 100%.

In some embodiments, gene disruption may occur by deletion of a genomic sequence using two guide RNAs. Methods of using CRISPR-Cas gene editing technology to create a genomic deletion in a cell (e.g., to knock out a gene in a cell) are known (Bauer D E et al. Vis. Exp. 2015; 95; e52118).

### 3. Spacer Sequence

In some embodiments, a gRNA comprises a spacer sequence. A spacer sequence is a sequence (e.g., a 20 nucleotide sequence) that defines the target sequence (e.g., a DNA target sequences, such as a genomic target sequence) of a target nucleic acid of interest. In some embodiments, the spacer sequence is 15 to 30 nucleotides. In some embodiments, the spacer sequence is 15, 16, 17, 18, 19, 29, 21, 22, 23, 24, 25, 26, 27, 28, 29, or 30 nucleotides. In some embodiments, a spacer sequence is 20 nucleotides.

The “target sequence” is adjacent to a PAM sequence and is the sequence modified by an RNA-guided nuclease (e.g., Cas9). The “target nucleic acid” is a double-stranded molecule: one strand comprises the target sequence and is referred to as the “PAM strand,” and the other complementary strand is referred to as the “non-PAM strand.” One of skill in the art recognizes that the gRNA spacer sequence hybridizes to the reverse complement of the target sequence, which is located in the non-PAM strand of the target nucleic acid of interest. Thus, the gRNA spacer sequence is the RNA equivalent of the target sequence. For example, if the target sequence is 5′-AGAGCAACAGTGCTGTGGCC-3′ (SEQ ID NO: 32), then the gRNA spacer sequence is 5′-AGAGCAACAGUGCUGUGGCC-3′ (SEQ ID NO: 33). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM of the Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence comprises 20 nucleotides. In some embodiments, the target nucleic acid comprises less than 20 nucleotides. In some embodiments, the target nucleic acid comprises more than 20 nucleotides. In some embodiments, the target nucleic acid comprises at least: 5, 10, 15, 16, 17, 18, 19, 20, 21, 22, 23, 24, 25, 30 or more nucleotides. In some embodiments, the target nucleic acid comprises at most: 5, 10, 15, 16, 17, 18, 19, 20, 21, 22, 23, 24, 25, 30 or more nucleotides. In some embodiments, the target nucleic acid sequence comprises 20 bases immediately 5′ of the first nucleotide of the PAM. For example, in a sequence comprising 5′-NNNNNNNNNNNNNNNNNNNNNRG-3′, the target nucleic acid comprises the sequence that corresponds to the Ns, wherein N is any nucleotide, and the underlined NRG sequence is the S. pyogenes PAM.

4. Methods of Making gRNAs

The gRNAs of the present disclosure are produced by a suitable means available in the art, including but not limited to in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in International Application PCT/US2013/30062. Accordingly, the present disclosure also includes polynucleotides, e.g., DNA, constructs and vectors are used to in vitro transcribe a gRNA described herein.

In some embodiments, non-natural modified nucleobases are introduced into polynucleotides, e.g., gRNA, during synthesis or post-synthesis. In certain embodiments, modifications are on internucleoside linkages, purine or pyrimidine bases, or sugar. In some embodiments, a modification is introduced at the terminal of a polynucleotide; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

In some embodiments, enzymatic or chemical ligation methods are used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

Certain embodiments of the invention also provide nucleic acids, e.g., vectors, encoding gRNAs described herein. In some embodiments, the nucleic acid is a DNA molecule. In other embodiments, the nucleic acid is an RNA molecule. In some embodiments, the nucleic acid comprises a nucleotide sequence encoding a crRNA. In some embodiments, the nucleotide sequence encoding the crRNA comprises a spacer flanked by all or a portion of a repeat sequence from a naturally-occurring CRISPR/Cas system. In some embodiments, the nucleic acid comprises a nucleotide sequence encoding a tracrRNA. In some embodiments, the crRNA and the tracrRNA is encoded by two separate nucleic acids. In other embodiments, the crRNA and the tracrRNA is encoded by a single nucleic acid. In some embodiments, the crRNA and the tracrRNA is encoded by opposite strands of a single nucleic acid. In other embodiments, the crRNA and the tracrRNA is encoded by the same strand of a single nucleic acid.

In some embodiments, the gRNAs provided by the disclosure are chemically synthesized by any means described in the art (see e.g., WO/2005/01248). While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together.

In some embodiments, the gRNAs provided by the disclosure are synthesized by enzymatic methods (e.g., in vitro transcription, IVT).

Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In certain embodiments, more than one guide RNA can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

The guide RNA may target any sequence of interest via the targeting sequence (e.g., spacer sequence) of the crRNA. In some embodiments, the degree of complementarity between the targeting sequence of the guide RNA and the target sequence on the target nucleic acid molecule is about 60%, 65%, 70%, 75%, 80%, 85%, 90%, 95%, 97%, 98%, 99%, or 100%. In some embodiments, the targeting sequence of the guide RNA and the target sequence on the target nucleic acid molecule is 100% complementary. In other embodiments, the targeting sequence of the guide RNA and the target sequence on the target nucleic acid molecule may contain at least one mismatch. For example, the targeting sequence of the guide RNA and the target sequence on the target nucleic acid molecule may contain 1, 2, 3, 4, 5, 6, 7, 8, 9, or 10 mismatches. In some embodiments, the targeting sequence of the guide RNA and the target sequence on the target nucleic acid molecule may contain 1-6 mismatches. In some embodiments, the targeting sequence of the guide RNA and the target sequence on the target nucleic acid molecule may contain 5 or 6 mismatches.

The length of the targeting sequence may depend on the CRISPR/Cas9 system and components used. For example, different Cas9 proteins from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence may comprise 18-24 nucleotides in length. In some embodiments, the targeting sequence may comprise 19-21 nucleotides in length. In some embodiments, the targeting sequence may comprise 20 nucleotides in length.

In some embodiments of the present disclosure, a CRISPR/Cas nuclease system includes at least one guide RNA. In some embodiments, the guide RNA and the Cas protein may form a ribonucleoprotein (RNP), e.g., a CRISPR/Cas complex. The guide RNA may guide the Cas protein to a target sequence on a target nucleic acid molecule (e.g., a genomic DNA molecule), where the Cas protein cleaves the target nucleic acid. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex.

5. Delivery of guide RNA and Nuclease

In some embodiments, a gRNA and an RNA-guided nuclease are delivered to a cell separately, either simultaneously or sequentially. In some embodiments, a gRNA and an RNA-guided nuclease are delivered to a cell together. In some embodiments, a gRNA and an RNA-guided nuclease are pre-complexed together to form a ribonucleoprotein (RNP).

RNPs are useful for gene editing, at least because they minimize the risk of promiscuous interactions in a nucleic acid-rich cellular environment and protect the RNA from degradation. Methods for forming RNPs are known in the art. In some embodiments, an RNP containing an RNA-guided nuclease (e.g., a Cas nuclease, such as a Cas9 nuclease) and a gRNA targeting a gene of interest is delivered a cell (e.g.: a T cell). In some embodiments, an RNP is delivered to a T cell by electroporation.

As used herein, a “β2M targeting RNP” refers to a gRNA that targets the β2M gene pre-complexed with an RNA-guided nuclease. In some embodiments, a β2M targeting RNP is delivered to a cell. In some embodiments, more than one RNP is delivered to a cell. In some embodiments, more than one RNA is delivered to a cell separately. In some embodiments, more than one RNP is delivered to the cell simultaneously.

In some embodiments, an RNA-guided nuclease is delivered to a cell in a DNA vector that expresses the RNA-guided nuclease, an RNA that encodes the RNA-guided nuclease, or a protein. In some embodiments, a gRNA targeting a gene is delivered to a cell as an RNA, or a DNA vector that expresses the gRNA.

Delivery of an RNA-guided nuclease, gRNA, and/or an RNP may be through direct injection or cell transfection using known methods, for example, electroporation or chemical transfection. Other cell transfection methods may be used.

### 6. Multi-Modal or Differential Delivery of Components

Skilled artisans will appreciate that different components of genome editing systems can be delivered together or separately and simultaneously or nonsimultaneously. Separate and/or asynchronous delivery of genome editing system components may be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., a RNA-guided nuclease molecule, gRNA, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components of a genome editing system, e.g., a RNA-guided nuclease and a gRNA, can be delivered by modes that differ in terms of resulting half-life or persistent of the delivered component the body, or in a particular compartment, tissue or organ. In an embodiment, a gRNA can be delivered by such modes. The RNA-guided nuclease molecule component can be delivered by a mode which results in less persistence or less exposure to the body or a particular compartment or tissue or organ.

More generally, in an embodiment, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV, adenovirus or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV, adenovirus or lentivirus. Transcription of these genes would be of little physiological consequence because the genes do not encode for a protein product, and the gRNAs are incapable of acting in isolation. The second component, a RNA-guided nuclease molecule, is delivered in a transient manner, for example as mRNA encoding the protein or as protein, ensuring that the full RNA-guided nuclease molecule/gRNA complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by WIC molecules. A two-part delivery system can alleviate these drawbacks.

Differential delivery modes can be used to deliver components to different, but overlapping target regions. The formation active complex is minimized outside the overlap of the target regions. Thus, in an embodiment, a first component, e.g., a gRNA is delivered by a first delivery mode that results in a first spatial, e.g., tissue, distribution. A second component, e.g., a RNA-guided nuclease molecule is delivered by a second delivery mode that results in a second spatial, e.g., tissue, distribution. In an embodiment the first mode comprises a first element selected from a liposome, nanoparticle, e.g., polymeric nanoparticle, and a nucleic acid, e.g., viral vector. The second mode comprises a second element selected from the group. In an embodiment, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA and the RNA-guided nuclease molecule are packaged in separated delivery vehicles with distinct but overlapping tissue tropism, the fully functional complex is only be formed in the tissue that is targeted by both vectors.

Iii. Knock-Down and/or Insertion into the P2M Loci of Human B Cells

In various embodiments, the invention relates to a population of cells comprising engineered human B cells, wherein the engineered human B cells comprise a disrupted β2M gene. In various embodiments, the β2M gene to be disrupted comprises SEQ ID NO. 1. In various embodiments, the β2M gene to be disrupted is at least 75%, 80%, 85%, 90%, 95% or 100% identical to the nucleic acid sequence of SEQ ID NO. 1.

In various embodiments, the disruption in the B2M gene results in an eliminated or decreased expression of the B2M gene. In various embodiments, expression of the β2M gene is reduced by 10%, 15%, 20%, 25%, 30%, 35%, 40%, 45%, 50%, 55%, 60%, 65%, 70%, 75%, 80%, 85%, 90%, 95%, or 100%. In preferred embodiments, β2M expression is reduced by at least 85%.

In various embodiments, the β2M gene is disrupted by deletion of all or part of the β2M gene. In various embodiments the β2M gene is disrupted by insertion of the gene encoding a therapeutic protein into a coding exon of the β2M gene. In various embodiments, the gene encoding a therapeutic protein (which is described in more detail below) is inserted into exon 2 of the β2M gene. In various embodiments, the gene encoding a therapeutic protein is inserted into exon 1 of the β2M gene. In various embodiments, the gene encoding a therapeutic protein is inserted into exon 3 of the β2M gene. In various embodiments, the gene encoding a therapeutic protein is inserted into exon 4 of the β2M gene.

In various embodiments, the gene encoding a therapeutic protein is inserted into an intron of the β2M gene. In various embodiments, the insertion of the gene encoding a therapeutic protein does not disrupt the expression of the B2M gene in a B cell.

Iv. Therapeutic Proteins

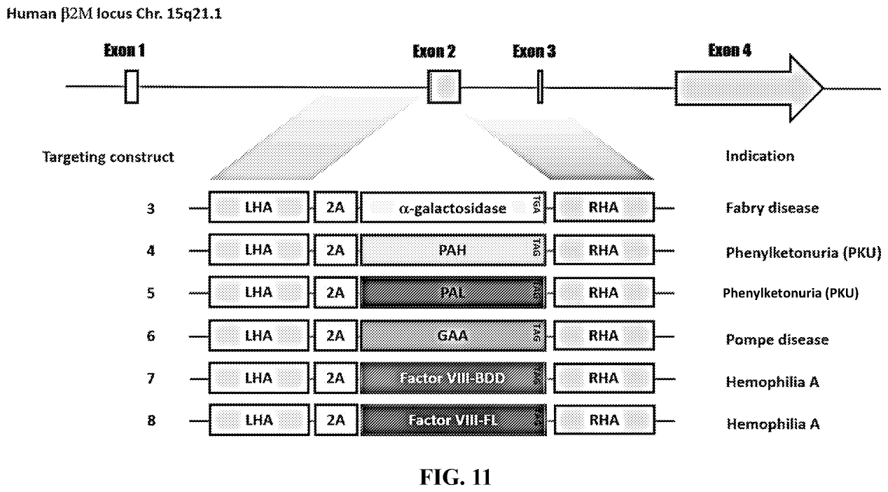

In various embodiments, the engineered B cell comprises a therapeutic protein to be delivered to a patient in need thereof. See for example FIG. 11, for a non-exclusive list of targeting constructs capable of expressing a therapeutic protein. As used herein, the term “therapeutic protein” means any protein that may contribute to the treatment, reduction of symptoms, prevention or cure of a disease or disorder in a patient. In certain embodiments, the therapeutic protein may be suitable for treatment of a rare disease or an orphan disease, where said therapy can be achieved by the replacement of a particular protein and/or enzyme. A therapeutic protein may include but is not limited to an enzyme, a ligand, a naturally occurring, engineered and/or chimeric receptor, a cytokine or a chemokine. Such disease include for example, but are not limited to Fabry disease, Pompe disease, Phenylketonuria (PKU) or Hemophilia A.

In various embodiments said therapeutic protein is a protein for the treatment of Fabry disease. In various embodiments, the therapeutic protein is a-galactosidase. In various embodiments, the therapeutic protein comprises the amino acid sequence of SEQ ID NO. 2. In various embodiments, the therapeutic protein is at least 75%, 80%, 85%, 90% or 95% identical to the amino acid sequence of SEQ ID NO. 2. In various embodiments, the targeting construct which comprises a left homology arm, a 2A cleavable peptide, a codon-optimized therapeutic protein and a right homology arm. In various embodiments, the targeting construct comprises the nucleic acid sequence of SEQ ID NO. 10. In various embodiments, the targeting construct is at least 75%, 80%, 85%, 90% or 95% identical to the codon-optimized nucleic acid sequence of SEQ ID NO. 10. In various embodiments, the targeting construct comprises the nucleic acid sequence of SEQ ID NO. 31. In various embodiments, the targeting construct is at least 75%, 80%, 85%, 90% or 95% identical to the nucleic acid sequence of SEQ ID NO. 31.

In various embodiments said therapeutic protein is a protein for the treatment of Phenylketonuria (PKU). In various embodiments, the therapeutic protein is phenylalanine hydroxylase (PAH). In various embodiments, the therapeutic protein comprises the amino acid sequence of SEQ ID NO. 3. In various embodiments, the therapeutic protein is at least 75%, 80%, 85%, 90% or 95% identical to the amino acid sequence of SEQ ID NO. 3. In various embodiments, the targeting construct which comprises a left homology arm, a 2A cleavable peptide, a codon-optimized therapeutic protein and a right homology arm comprises the nucleic acid sequence of SEQ ID NO. 11. In various embodiments, the targeting construct is at least 75%, 80%, 85%, 90% or 95% identical to the nucleic acid sequence of SEQ ID NO. 11. In various embodiments, the therapeutic protein is phenylalanine ammonia-lyase (PAL). In various embodiments, the therapeutic protein comprises the codon-optimized amino acid sequence of SEQ ID NO. 4. In various embodiments, the therapeutic protein is at least 75%, 80%, 85%, 90% or 95% identical to the amino acid sequence of SEQ ID NO. 4. In various embodiments, the targeting construct which comprises a left homology arm, a 2A cleavable peptide, a codon-optimized therapeutic protein and a right homology arm comprises the nucleic acid sequence of SEQ ID NO. 12. In various embodiments, the targeting construct is at least 75%, 80%, 85%, 90% or 95% identical to the nucleic acid sequence of SEQ ID NO. 12.

In various embodiments said therapeutic protein is a protein for the treatment of Pompe disease. In various embodiments, the therapeutic protein is acid alpha-glucosidase (GAA). In various embodiments, the therapeutic protein comprises the amino acid sequence of SEQ ID NO. 5. In various embodiments, the therapeutic protein is at least 75%, 80%, 85%, 90% or 95% identical to the amino acid sequence of SEQ ID NO. 5. In various embodiments, the targeting construct which comprises a left homology arm, a 2A cleavable peptide, a codon-optimized therapeutic protein and a right homology arm comprises the nucleic acid sequence of SEQ ID NO. 13. In various embodiments, the targeting construct is at least 75%, 80%, 85%, 90% or 95% identical to the nucleic acid sequence of SEQ ID NO. 13.

In various embodiments said therapeutic protein is a protein for the treatment of Hemophilia A. In various embodiments, the therapeutic protein is B domain deleted (BDD) of Factor VIII. In various embodiments, the therapeutic protein comprises the amino acid sequence of SEQ ID NO. 6. In various embodiments, the therapeutic protein is at least 75%, 80%, 85%, 90% or 95% identical to the amino acid sequence of SEQ ID NO. 6. In various embodiments, In various embodiments, the targeting construct which comprises a left homology arm, a 2A cleavable peptide, a codon-optimized therapeutic protein and a right homology arm comprises the nucleic acid sequence of SEQ ID NO. 14. In various embodiments, the targeting construct is at least 75%, 80%, 85%, 90% or 95% identical to the codon-optimized nucleic acid sequence of SEQ ID NO. 14. In various embodiments, the therapeutic protein is the full length domain of Factor VIII. In various embodiments, the therapeutic protein comprises the amino acid sequence of SEQ ID NO. 7. In various embodiments, the therapeutic protein is at least 75%, 80%, 85%, 90% or 95% identical to the amino acid sequence of SEQ ID NO. 7. In various embodiments, the targeting construct which comprises a left homology arm, a 2A cleavable peptide, a codon-optimized therapeutic protein and a right homology arm comprises the nucleic acid sequence of SEQ ID NO. 15. In various embodiments, the targeting construct is at least 75%, 80%, 85%, 90% or 95% identical to the nucleic acid sequence of SEQ ID NO. 15.

In various embodiments, the therapeutic protein is a chimeric receptor that expresses an extracellular domain of GPC3. In various embodiments, the therapeutic protein comprises the amino acid sequence of SEQ ID NO. 8. In various embodiments, the therapeutic protein is at least 75%, 80%, 85%, 90% or 95% identical to the amino acid sequence of SEQ ID NO. 8. In various embodiments, the targeting construct which comprises a left homology arm, a 2A cleavable peptide, a codon-optimized therapeutic protein and a right homology arm comprises the nucleic acid sequence of SEQ ID NO. 16. In various embodiments, the targeting construct is at least 75%, 80%, 85%, 90% or 95% identical to the nucleic acid sequence of SEQ ID NO. 16.

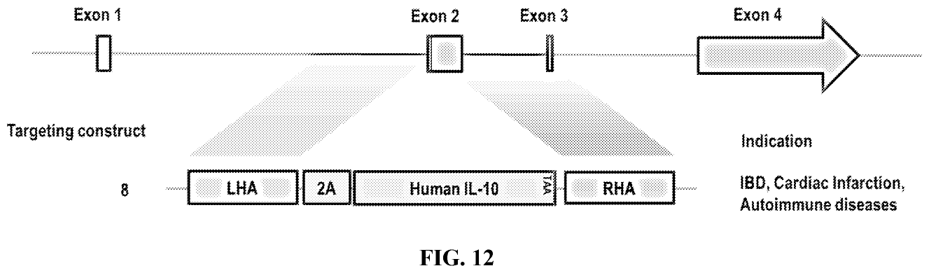

In various embodiments, the therapeutic protein is Interleukin 10 (IL-10). See for example FIG. 12. In various embodiments, the therapeutic protein comprises the amino acid sequence of SEQ ID NO. 9. In various embodiments, the therapeutic protein is at least 75%, 80%, 85%, 90% or 95% identical to the amino acid sequence of SEQ ID NO. 9. In various embodiments, the targeting construct which comprises a left homology arm, a 2A cleavable peptide, a codon-optimized therapeutic protein and a right homology arm comprises the nucleic acid sequence of SEQ ID NO. 17. In various embodiments, the targeting construct is at least 75%, 80%, 85%, 90% or 95% identical to the nucleic acid sequence of SEQ ID NO. 17.

In various embodiments, the present disclosure relates to a method of expressing a therapeutic protein in a population of human B cells. In various embodiments, at least 20% of the human B cells express the therapeutic protein. In various embodiments at least 10%, 15%, 20%, 25%, 30%, 35%, 40%, 45%, 50%, 55%, 60%, 65%, 70%, 75%, 80%, 85%, 90% or 95% of the engineered B cells express the therapeutic protein.

### V. Methods of Treatment

In various aspects of the invention, the gene edited B cells will be delivered as a therapeutic to a patient in need thereof. In various embodiments, the gene edited B cells will be capable of treating or preventing various diseases or disorders.

In some embodiments, are methods for treating a rare disease or an orphan disease, where said therapy can be achieved by the replacement of a particular protein and/or enzyme. Such diseases include for example, but are not limited to Fabry disease, Pompe disease, Phenylketonuria (PKU) or Hemophilia A.

In some aspects, the invention comprises a pharmaceutical composition comprising a population of gene edited B cells comprising at least one therapeutic protein as described herein and a pharmaceutically acceptable excipient. In some embodiments, the pharmaceutical composition further comprises an additional active agent.

It will be appreciated that target doses for modified B cells can range from 1×10⁶-2×10¹⁰ cells/kg, preferably 2×10⁶ cells/kg, more preferably. It will be appreciated that doses above and below this range may be appropriate for certain subjects, and appropriate dose levels can be determined by the healthcare provider as needed. Additionally, multiple doses of cells can be provided in accordance with the invention.

In some embodiments, the expanded population of engineered B cells are autologous B cells. In some embodiments, the modified B cells are allogeneic B cells. In some embodiments, the modified B cells are heterologous B cells. In some embodiments, the modified B cells of the present application are transfected or transduced in vivo. In other embodiments, the engineered cells are transfected or transduced ex vivo.

As used herein, the term “subject” or “patient” means an individual. In some aspect, a subject is a mammal such as a human. In some aspect, a subject can be a non-human primate. Non-human primates include marmosets, monkeys, chimpanzees, gorillas, orangutans, and gibbons, to name a few. The term “subject” also includes domesticated animals, such as cats, dogs, etc., livestock (e.g., llama, horses, cows), wild animals (e.g., deer, elk, moose, etc.,), laboratory animals (e.g., mouse, rabbit, rat, gerbil, guinea pig, etc.) and avian species (e.g., chickens, turkeys, ducks, etc.). Preferably, the subject is a human subject. More preferably, the subject is a human patient.

In certain embodiments, compositions comprising gene edited B cells disclosed herein may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, que-lamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as TARGRETIN™ (bexarotene), PANRETIN™, (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (CYTOXAN®) Doxorubicin (hydroxydoxorubicin), Fludarabine, Vincristine (ONCOVIN®), and Prednisone.

A variety of additional therapeutic agents may be used in conjunction with the compositions described herein. For example, potentially useful additional therapeutic agents include PD-1 (or PD-L1) inhibitors such as nivolumab (Opdivo®), pembrolizumab (Keytruda®), pembrolizumab, cemiplimaib (Libtayo®), and atezolizumab (Tecentriq®). Other additional therapeutics include anti-CTLA-4 antibodies (e.g., Ipilimumab®), anti-LAG-3 antibodies (e.g., Relatlimab, BMS), alone or in combination with PD-1 and/or PD-L1 inhibitors.

Additional therapeutic agents suitable for use in combination with the invention include, but are not limited to, ibrutinib (IMBRUVICA®), ofatumumab (ARZERRA®), rituximab (RITUXAN®), bevacizumab (AVASTIN®), trastuzumab (HERCEPTIN®), trastuzumab emtansine (KADCYLA®), imatinib (GLEEVEC®), cetuximab (ERBITUX®), panitumumab (VECTIBIX®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In additional embodiments, the composition comprising gene edited B cells can be administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®)), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the compositions described herein are administered in conjunction with a cytokine. “Cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and —II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1 alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

### EXAMPLES

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### Example 1. Engineering Human B Cells—Optimal Nucleofection Conditions

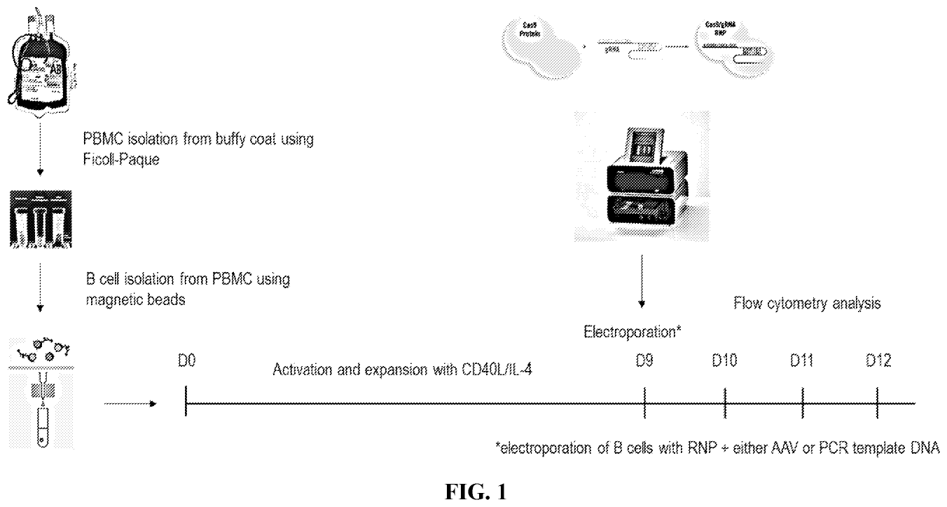

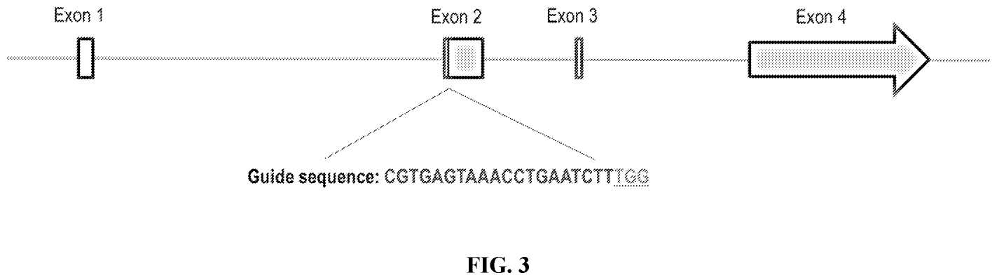

The experimental design for the below described experiments is outlined in FIG. 1. Briefly, PBMCs were isolated from healthy donors using magnetic beads and activated using CD40 ligand and IL4. B cells were engineered using nucleofection delivery of the CRISPR-Cas9 system and resulting engineered B cells were characterized by PCR and flow cytometry. FIG. 3 depicts the insertion site in the β2M gene and guide sequence used for editing.

Human B cell isolation, activation, expansion and electroporation. Buffy coats from healthy donors were obtained from Stanford Blood Center (Menlo Park, CA, USA). PBMCs were isolated from buffy coats using Ficoll-Paque (GE Healthcare, Chicago, IL). Primary human B cells were isolated using the EASYSEP™ Human B Cell Isolation Kit according to manufacturer's instruction (STEMCELL Technologies Inc., Cambridge, MA, USA). Isolated B cells were activated and expanded using the human B Cell Expansion Kit according to manufacturer's instruction (Miltenyi Biotec, Bergisch Gladbach, Germany).

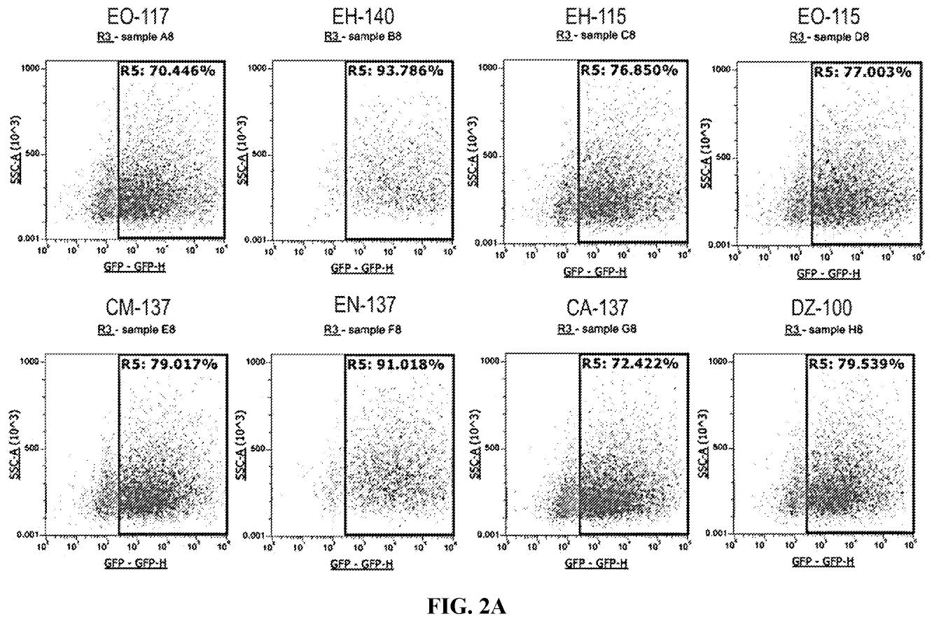

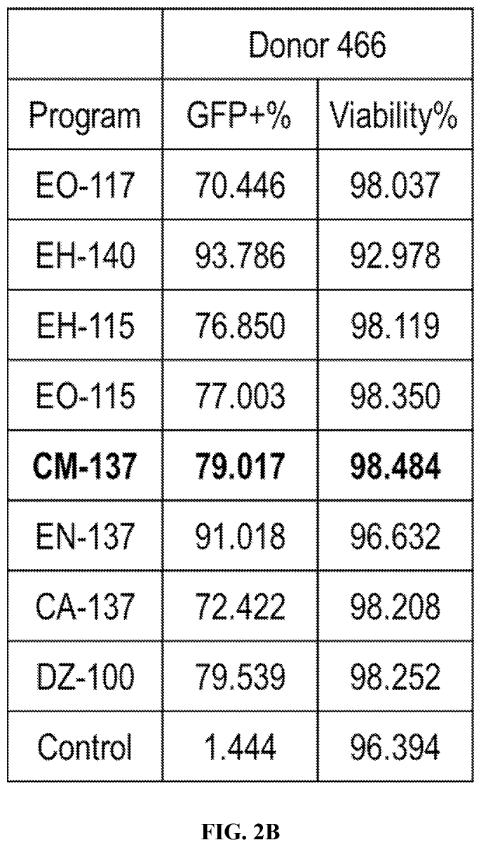

Optimal Nucleofection Protocol Development. Nucleofection was performed using AMAXA™ 4D-NUCLEOFACTOR™ in P3 nucleofection solution (Lonza, Basel, Switzerland). 1 μg of pMAX-GFP plasmid DNA was used to electroporate 1 million activated human B cells in 20 μl volume for GFP expression. Various electroporation programs were examined both for efficiency of transfection (FIG. 2A and FIG. 2B (“GFP+%”)) and for the percentage of viable cells achieved (FIG. 2A and FIG. 2B (“Viability %”)).

Results. It was determined that program CM-137 achieved the most optimal combination of efficiency (79.017%) and viability (98.484%).

### Example 2. Optimization of CRISPR Engineering Conditions

Next, parameters for delivery of the CRISPR-Cas9 complex were explored. PBMC-derived human B cells were isolated, activated and expanded as described in Example 1. Next, β2M targeting Cas9/sgRNA RNPs were prepared and electroporated into the B-cells. β2M knock-down and B-cell viability were evaluated.

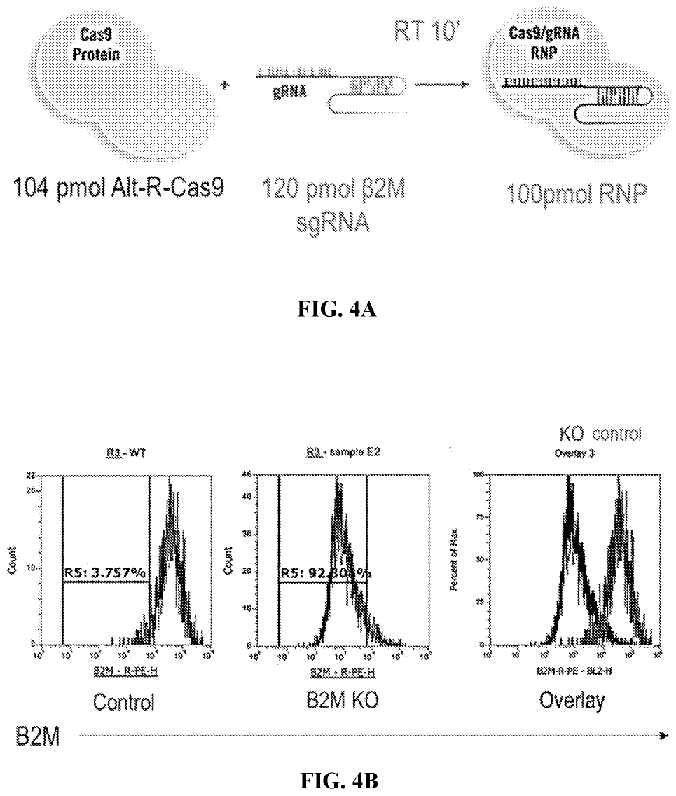

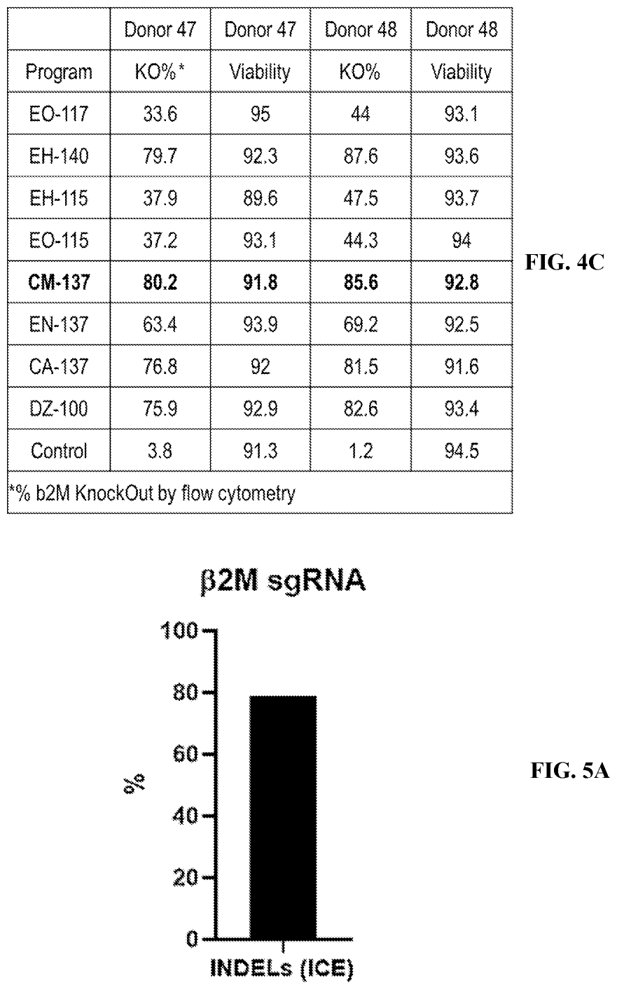

β2M sgRNA and CRISPR engineering. A chemically modified sgRNA oligomer targeting β 2M was manufactured by IDT (Integrated DNA Technologies, Coralville, Iowa, USA). See, e.g., FIG. 3. Recombinant S. pyogenes Cas9 enzyme was purchased from IDT (Integrated DNA Technologies, Coralville, Iowa, USA). Cas9 was incubated with sgRNA at a molar ratio of 1:1.2 at room temperature for 10 minutes prior to mixing with B cells. 100 pmol RNP was used for electroporation with 1 million activated human B cells in 20 μl volume (FIG. 4A). Engineering of primary human B cells was carried out using an AMAXA™ 4D-NUCLEOFACTOR™ in P3 nucleofection solution with one of the 8 preset programs (Lonza, Basel, Switzerland). β2M knock-down and viability were assessed using flow cytometry.

Results. It was determined that program CM-137 achieved the most optimal combination of β2M knock-down (80.2% (donor 47) and 91.8% (donor 48)) and viability (85.6% (donor 47) and 92.8% (donor 48)). See, e.g., FIG. 4C.

### Example 3. P2M CRISPR Knockout INDELs Analysis

Next, the knockout of the β2M by CRISPR/cas9 was validated through INDEL analysis. PBMC-derived human B cells were isolated, activated and expanded as described in Example 1. Next, β2M targeting Cas9/sgRNA RNPs were prepared and electroporated into the B-cells.

Engineered cells were cultured for 2 days after electroporation. Genomic DNA was extracted using NucleoSpin Tissue, Mini kit for DNA from cells and tissue polymerase (Thermo Fisher Scientific, Waltham, MA, USA), according to the manufacturer's recommendations. To interrogate the sites of DNA cleavage after editing, PCR was performed using Q5 High-Fidelity polymerase (VWR International, LLC, Radnor, PA, USA) and primers flanking the region where double stranded breaks were generated. The PCR amplicons were purified using QIAquick PCR Purification Kit (Qiagen, Hilden, Germany) and sequenced by Sanger sequencing. The resulting sequences were used to calculate INDELs frequencies using ICE synthego (ice.synthego.com) web-based software. A list of the primer sequences is provided in Table 2.

TABLE 2
Primers for INDELs analysis
of β2M specific sgRNA.

| Primer ID | Sequence |
|---|---|
| InDels-B2M-FWD | TGAGAGGGCATCAGAAGTCC |
| (SEQ ID NO: 27) | |
| InDels-B2M-Rev | AAGTCACATGGTTCACACGG |
| (SEQ ID NO: 28) | |

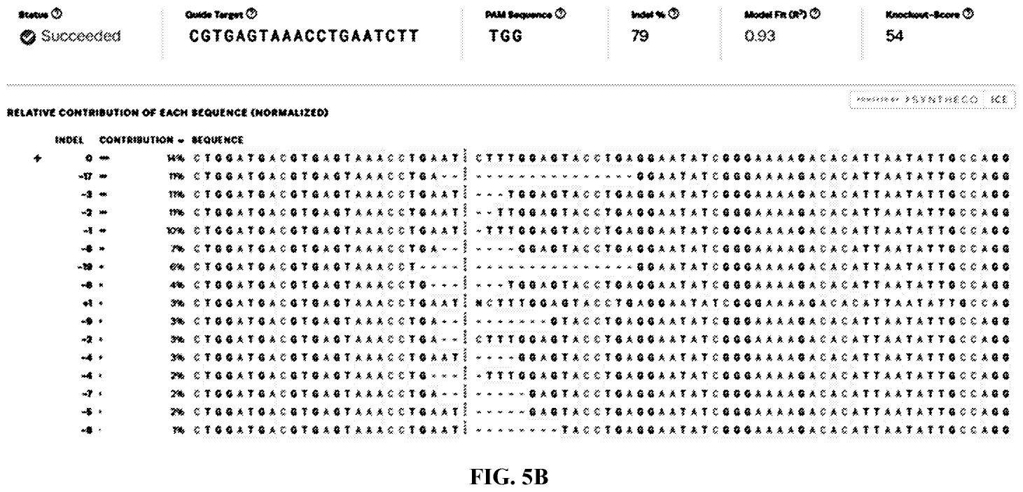

The results are depicted as FIG. 5. FIG. 5A shows the quantification of insertions and deletions generated at the cut site of the β2M sgRNA. Genomic DNA was extracted from B cells nucleofected with Cas9 and β2M sgRNA 2 days after editing and Sanger sequencing was performed to quantify INDELs at the cut site. FIG. 5B shows an overview of the insertions and deletions generated at the cut site of the β2M sgRNA.

### Example 4. Genome Targeting with rAAV6 and Assessment of HDR-Mediated Targeted Integration

Next, genome targeting with an rAAV6 expressing either green fluorescent protein (GFP) or GPC3-CAR HDR donor cassettes was assessed for HDR-mediated targeted integration.

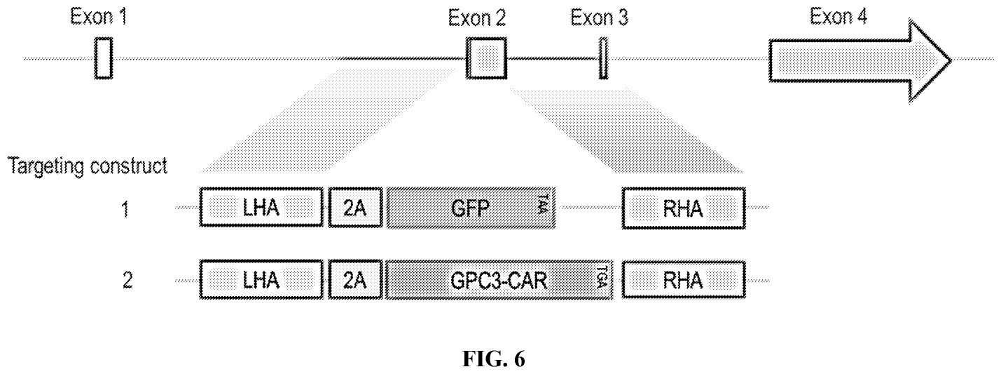

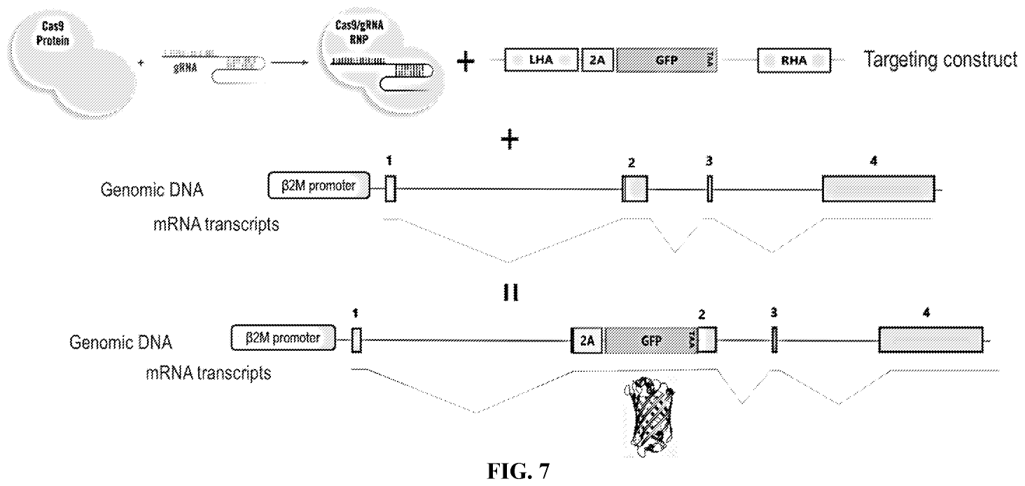

β2M-targeting constructs. β2M-targeting constructs (either GFP (SEQ ID NO:25) or GPC3-CAR (SEQ ID NO. 16)) were synthesized by Genscript (Piscataway, NJ, USA), and cloned into pAAV6 vector (CellBiolabs, San Diego, CA, USA). rAAV6 viruses were produced by Vigene Biosciences (Rockville, MD, USA). See, e.g., FIGS. 6 and 7.

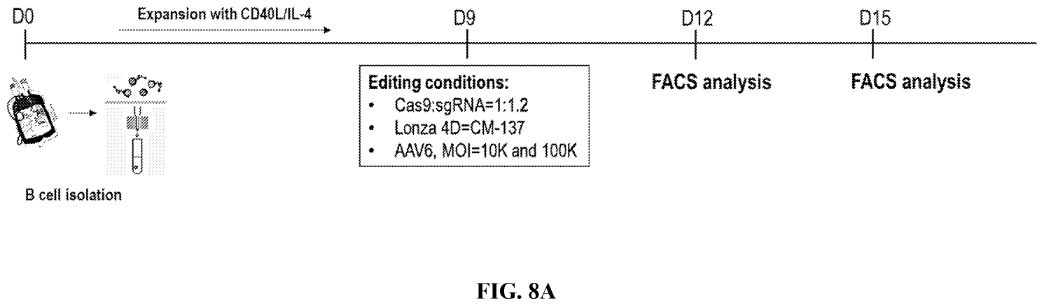

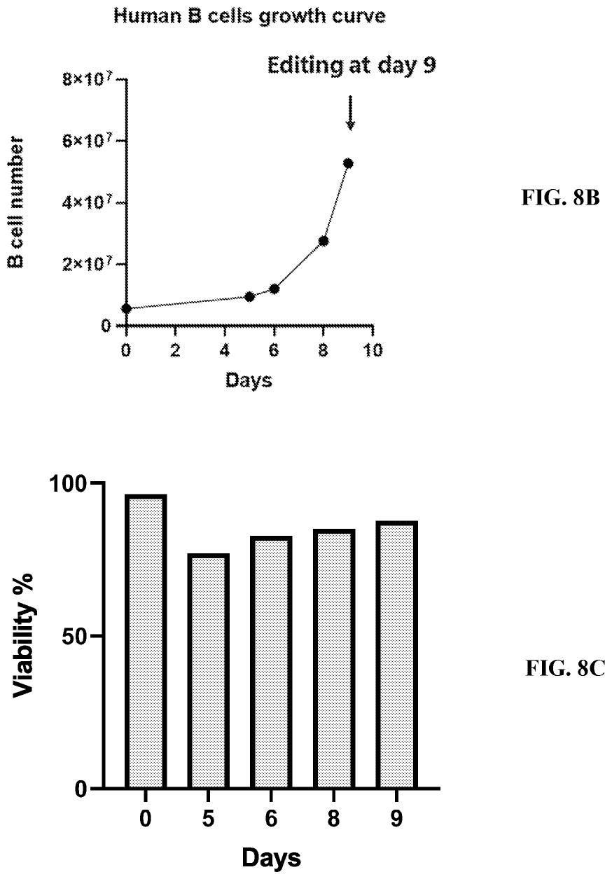

Human B cell isolation, activation, expansion and electroporation. The experimental design for the below described experiments is outlined in FIG. 8A. First, primary human B cells were isolated, activated and as described in Example 1 and 2 above. The growth curve of the cultured human B cells (FIG. 8B) and the viability of cultured human B cells was evaluated over the course of expansion (FIG. 8C). At day 9, gene editing was performed as described below.

Nucleofection Transduction. B cells were first nucleofected with the β2M-specific RNP using the protocol described in Examples 1 and 2, and then immediately transduced with AAV6 donor at a multiplicity of infection (MOI) of 10,000 viral genomes (vg)/μl or 100,000 vg/μl to maximize efficiency of transduction (Bak et al., 2018; Charlesworth et al., 2018). B cells were cultured as for an additional 3 or 6 days and efficiency of integration was assessed.

Efficiency and Integration. Rates of targeted integration of the GFP and GPC3 donors were measured by flow cytometry 3 or 6 days after electroporation and AAV6 transduction. Targeted integration of the GFP and GPC3 expression cassettes was measured by flow cytometry using Attune NxT Flow Cytometer (Invitrogen, Carlsbad, CA, USA). GPC3-CAR was detected using a biotinylated human Glypican 3 with His and Avi-tag (GP3-H82E5, Acro Biosystem, Newark, DE, USA), conjugated to a BV421-labeled streptavidin (Biolegend, San Diego, CA, USA). Additionally, cells were stained with LIVE/DEAD™ Fixable Near-IR (Invitrogen, Carlsbad, CA, USA) to discriminate live and dead cells according to manufacturer's instructions.

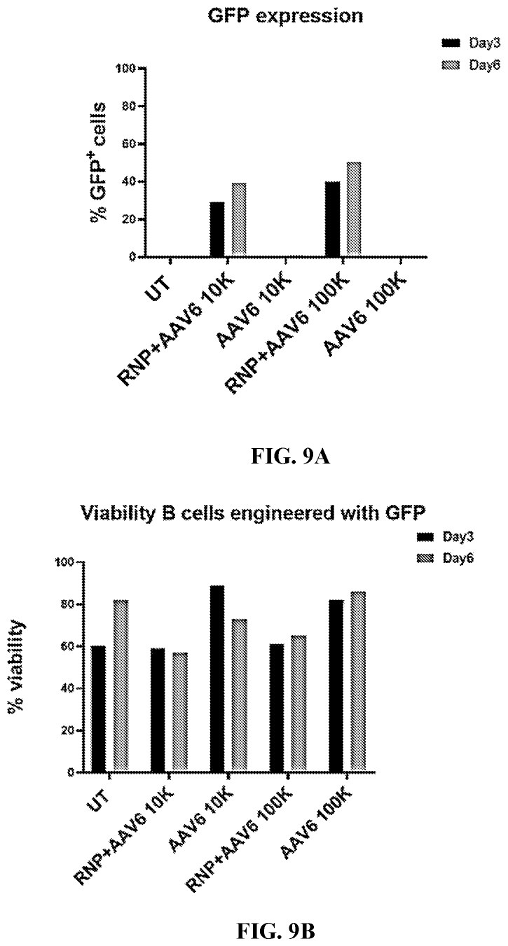

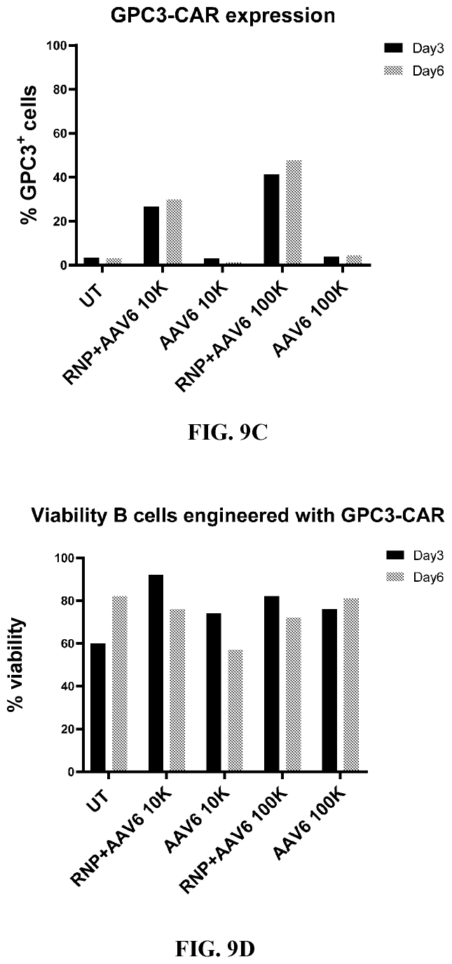

Results. Promoter-less GFP targeting constructs encoded in an AAV6 virus were efficiently integrated into the B2M locus in activated human B cells, leading to protein expression detected by flow cytometry. See, e.g., FIGS. 9A and 9B. AAV6 mediated promoter-less GPC3-CAR targeting into the β2M locus achieved similar efficiencies in human B cells. FIGS. 9C and 9D. At MOI of 100K, the targeting efficiency was greater than 40% for both the GFP and GPC3 CAR constructs. FIGS. 9A and 9C. Further, there did not appear to be any reduction in B cell viability. FIGS. 9B and 9D.

### Example 5. Genome Targeting with dsRNA and Assessment of HDR-Mediated Targeted Integration

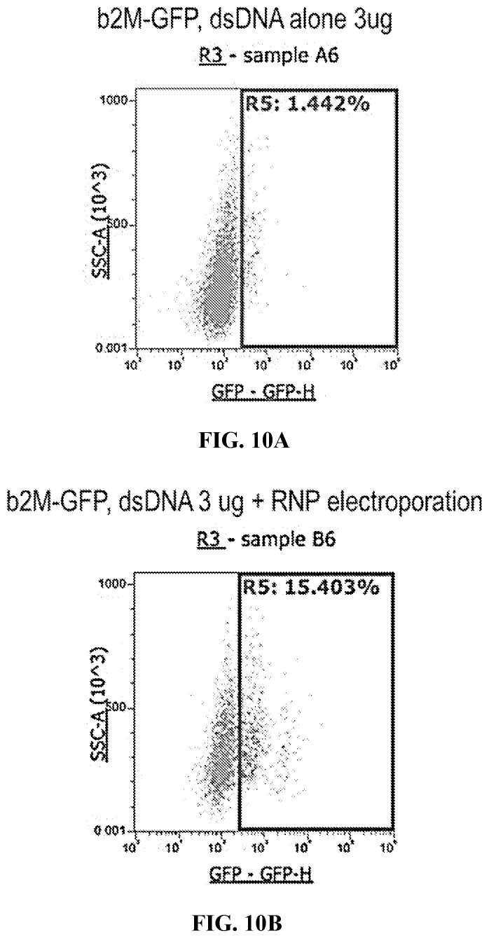

Next, genome targeting with a dsDNA HDR construct expressing green fluorescent protein (GFP) was assessed for HDR-mediated targeted integration. See, e.g., FIG. 10.

Human B cell isolation, activation, expansion and electroporation. First, primary human B cells were isolated, activated and as described in Example 1 and 2 above. The growth curve of the cultured human B cells and the viability of cultured human B cells was evaluated over the course of expansion. At day 9, gene editing was performed as described below.

dsRNA. For viral-free engineering, β2M-targeting constructs were amplified by PCR using Q5 High-Fidelity polymerase (VWR International, LLC, Radnor, PA, USA) with forward primer 5′-GCTATGTCCCAGGCACTCTAC-3′ (SEQ ID NO: 29) and reverse primer 5′-AGGATGCTAGGACAGCAGGA-3′ (SEQ ID NO: 30). PCR products were purified using NUCLEOSPIN® Gel and PCR Clean-Up kits (TaKaRa Bio, Mountain View, CA, USA).

Nucleofection Transduction. B cells were first nucleofected with the β2M-specific RNP using the protocol described in Examples 1 and 2.

Efficiency and Integration. Rates of targeted integration of the GFP and GPC3 donors were measured by flow cytometry 3 or 6 days after electroporation and AAV6 transduction. Targeted integration of the GFP and GPC3 expression cassettes was measured by flow cytometry using Attune NxT Flow Cytometer (Invitrogen, Carlsbad, CA, USA). GPC3-CAR was detected using a biotinylated human Glypican 3 with His and Avi-tag (GP3-H82E5, Acro Biosystem, Newark, DE, USA), conjugated to a BV421-labeled streptavidin (Biolegend, San Diego, CA, USA). Additionally, cells were stained with LIVE/DEAD™ Fixable Near-IR (Invitrogen, Carlsbad, CA, USA) to discriminate live and dead cells according to manufacturer's instructions.

Results. Promoter-less GFP targeting constructs encoded in a dsDNA were integrated into the β2M locus in activated human B cells, leading to protein expression detected by flow cytometry. See, e.g., FIG. 10. When electroporated with 3 μg of the dsDNA construct alone, no GFP expression was observed. FIG. 10A. But when the 3 μg of the dsDNA construct was electroporated with the RNP complex, significant GFP expression (indicative of integration into the β2M locus was observed. FIG. 10B.

### Example 6. Genome Targeting with rAAV6 and Assessment of GLA Expression in Engineered B Cells

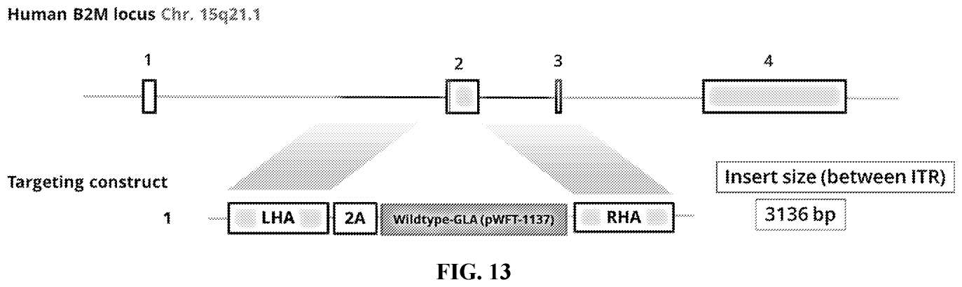

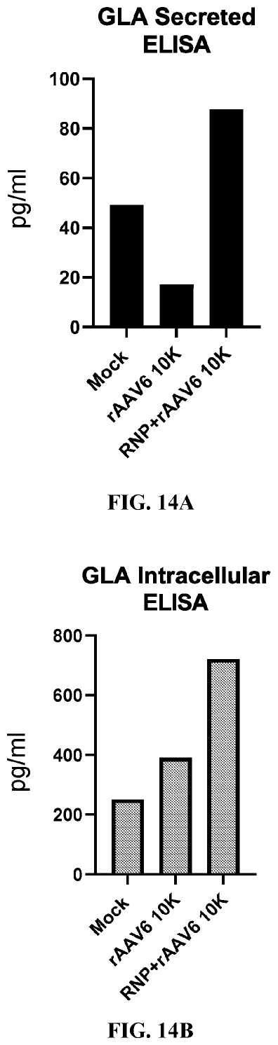

Next, genome targeting with an rAAV6 expressing the wild type GLA protein was assessed for in vitro GLA expression and secretion by the engineered B cells. See, e.g., FIGS. 13 and 14.

Human B cell isolation, activation, expansion and electroporation. First, primary human B cells were isolated, activated and as described in Example 1 and 2 above. At day 7, gene editing was performed as described below.

β2M-targeting constructs. β2M-targeting constructs (SEQ ID NO. 31) were synthesized by Genscript (Piscataway, NJ, USA), and cloned into pAAV6 vector (CellBiolabs, San Diego, CA, USA). rAAV6 viruses were produced by Vigene Biosciences (Rockville, MD, USA).

Nucleofection Transduction. B cells were first nucleofected with the β2M-specific RNP using the protocol described in Examples 1 and 2, and then immediately transduced with AAV6 donor at a multiplicity of infection (MOI) of 10,000 viral genomes (vg)/μl to maximize efficiency of transduction (Bak et al., 2018; Charlesworth et al., 2018). B cells were cultured as for an additional 5 days. Next, efficiency of integration was assessed using qualitative PCR and expression of GLA in the supernatant and B cell lysates using ELISA.

Efficiency of Integration And Transgene Expression. Rates of targeted integration of the GLA donors were measured by qualitative PCR 5 days after electroporation and AAV6 transduction. Intracellular and secreted GLA was measured using an ELISA assay. Results. Promoter-less GLA constructs encoded in an AAV6 virus were integrated into the B2M locus in activated human B cells with an efficiency of about 20 to 30%. B cells engineered with the Cas9 RNP-GLA rAAV6 demonstrated a significant increase in GLA expression intracellularly, as well as an increase in extracellular secretion of GLA.

1-78. (canceled)

79. An engineered human B cell comprising a therapeutic protein encoded by a nucleic acid sequence inserted into a locus of a β2M gene.

80. The engineered human B cell of claim 79, wherein expression of the β2M gene has been disrupted.

81. The engineered human B cell of claim 80, wherein the nucleic acid sequence encoding the therapeutic protein has been inserted into exon 2 of the locus of the β2M gene.

82. The engineered human B cell of claim 79, wherein the nucleic acid sequence encoding the therapeutic protein has been inserted into an intron of the locus of the β2M gene, and wherein expression of the β2M gene is maintained at a percentage greater than 50% when compared with a wild type human B cell.

83. The engineered human B cell of claim 79, wherein the therapeutic protein is alpha-galactosidase A (GLA), acid alpha-glucosidase (GAA), phenylalanine hydroxylase (PAH), phenylalanine ammonia-lyase (PAL), full length or B domain-deleted (BDD) FVIII, a GPC3 chimeric receptor, a cytokine or a chemokine.

84. The engineered human B cell of claim 82, wherein the therapeutic protein is selected from the amino acid sequences consisting of SEQ ID NOs. 2-9.

85. A method of producing an engineered B cell expressing a therapeutic protein, the method comprising delivering to a human B cell:
a. an RNA-guided nuclease;
b. a gRNA targeting a β2M gene; and
c. a construct comprising a nucleic acid sequence encoding the therapeutic protein.

86. The method of claim 85, wherein the RNA-guided nuclease comprises the amino acid sequence of SEQ ID NO. 18.

87. The method of claim 85, wherein the gRNA comprises the nucleic acid sequence of SEQ ID NO. 19.

88. The method of claim 85, wherein the gRNA specifically targets exon 2 of a locus of the β2M gene.

89. The method of claim 85, wherein the gRNA specifically targets an intron of a locus of the β2M gene.

90. The method of claim 85, wherein the β2M gene expresses at a percentage greater than 50% when compared to a wild-type human B cell.

91. The method of claim 85, wherein the construct comprises a codon-optimized nucleic acid sequence selected from the group consisting of SEQ ID NOs. 10-17 and 31.

92. The method of claim 85, wherein the construct comprises a left homology arm of SEQ ID NO. 20 and a right homology arm of SEQ ID NO. 21.

93. A method of treating a patient in need thereof comprising administering to said patient a therapeutically effective amount of an engineered human B cell, wherein the engineered human B cell comprises a therapeutic protein, wherein a gene expressing said therapeutic protein has been inserted into a locus of a β2M gene.

94. The method of claim 93, wherein the therapeutic protein is for the treatment of Fabry disease, Pompe disease, Phenylketonuria (PKU) or Hemophilia A.

95. The method of claim 94, wherein expression of the β2M gene has been disrupted.

96. The method of claim 95, wherein a nucleic acid sequence encoding the therapeutic protein has been inserted into exon 2 of the locus of the β2M gene.

97. The method of claim 94, wherein the therapeutic protein is alpha-galactosidase A (GLA), acid alpha-glucosidase (GAA), phenylalanine hydroxylase (PAH), phenylalanine ammonia-lyase (PAL) or B domain-deleted (BDD) FVIII.

98. The method of claim 97, wherein the therapeutic protein is selected from the amino acid sequences consisting of SEQ ID NOs. 2-7.


SEQUENCE LISTING (50 sequences)

SEQ ID NO: 1 - DNA, 7361 bp, Homo sapiens
attcctgaagctgacagcattcgggccgagatgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcatttttttttaagaaaaacgcctgccttctgcgtgagattctccagagcaaactgggcggcatgggccctgtggtcttttcgtacagagggcttcctctttggctctttgcctggttgtttccaagatgtactgtgcctcttactttcggttttgaaaacatgagggggttgggcgtggtagcttacgcctgtaatcccagcacttagggaggccgaggcgggaggatggcttgaggtccgtagttgagaccagcctggccaacatggtgaagcctggtctctacaaaaaataataacaaaaattagccgggtgtggtggctcgtgcctgtggtcccagctgctccggtggctgaggcgggaggatctcttgagcttaggcttttgagctatcatggcgccagtgcactccagcgtgggcaacagagcgagaccctgtctctcaaaaaagaaaaaaaaaaaaaaagaaagagaaaagaaaagaaagaaagaagtgaaggtttgtcagtcaggggagctgtaaaaccattaataaagataatccaagatggttaccaagactgttgaggacgccagagatcttgagcactttctaagtacctggcaatacactaagcgcgctcaccttttcctctggcaaaacatgatcgaaagcagaatgttttgatcatgagaaaattgcatttaatttgaatacaatttatttacaacataaaggataatgtatatatcaccaccattactggtatttgctggttatgttagatgtcattttaaaaaataacaatctgatatttaaaaaaaaatcttattttgaaaatttccaaagtaatacatgccatgcatagaccatttctggaagataccacaagaaacatgtaatgatgattgcctctgaaggtctattttcctcctctgacctgtgtgtgggttttgtttttgttttactgtgggcataaattaatttttcagttaagttttggaagcttaaataactctccaaaagtcataaagccagtaactggttgagcccaaattcaaacccagcctgtctgatacttgtcctcttcttagaaaagattacagtgatgctctcacaaaatcttgccgccttccctcaaacagagagttccaggcaggatgaatctgtgctctgatccctgaggcatttaatatgttcttattattagaagctcagatgcaaagagctctcttagcttttaatgttatgaaaaaaatcaggtcttcattagattccccaatccacctcttgatggggctagtagcctttccttaatgatagggtgtttctagagagatatatctggtcaaggtggcctggtactcctccttctccccacagcctcccagacaaggaggagtagctgccttttagtgatcatgtaccctgaatataagtgtatttaaaagaattttatacacatatatttagtgtcaatctgtatatttagtagcactaacacttctcttcattttcaatgaaaaatatagagtttataatattttcttcccacttccccatggatggtctagtcatgcctctcattttggaaagtactgtttctgaaacattaggcaatatattcccaacctggctagtttacagcaatcacctgtggatgctaattaaaacgcaaatcccactgtcacatgcattactccatttgatcataatggaaagtatgttctgtcccatttgccatagtcctcacctatccctgttgtattttatcgggtccaactcaaccatttaaggtatttgccagctcttgtatgcatttaggttttgtttctttgttttttagctcatgaaattaggtacaaagtcagagaggggtctggcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccattcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctcttatatctcactcccactattacccctttattttcaaacagggaaacagtcttcaagttccacttggtaaaaaatgtgaaccccttgtatatagagtttggctcacagtgtaaagggcctcagtgattcacattttccagattaggaatctgatgctcaaagaagttaaatggcatagttggggtgacacagctgtctagtgggaggccagccttctatattttagccagcgttctttcctgcgggccaggtcatgaggagtatgcagactctaagagggagcaaaagtatctgaaggatttaatattttagcaaggaatagatatacaatcatcccttggtctccctgggggattggtttcaggaccccttcttggacaccaaatctatggatatttaagtcccttctataaaatggtatagtatttgcatataacctatccacatcctcctgtatactttaaatcatttctagattacttgtaatacctaatacaatgtaaatgctatgcaaatagttgttattgtttaaggaataatgacaagaaaaaaaagtctgtacatgctcagtaaagacacaaccatccctttttttccccagtgtttttgatccatggtttgctgaatccacagatgtggagcccctggatacggaaggcccgctgtactttgaatgacaaataacagatttaaaattttcaaggcatagttttatacctga

SEQ ID NO: 2 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 3 - protein, 452 aa, Homo sapiens
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 4 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARASLSPATERLYSAVRHVVGQKPTSDRPYIWNDNEQGLDEHIARISADIAAGGVIVQAVQDILPSLH

SEQ ID NO: 5 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 6 - protein, 1457 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTVKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 7 - protein, 2351 aa, Artificial Sequence
  Description of Artificial Sequence Synthetic polypeptide
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 8 - protein, 416 aa, Artificial Sequence
  Description of Artificial Sequence Synthetic polypeptide
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSAAAFVPVFLPAKPTTTPAPRPPTPAPTIASQPLSLRPEACRPAAGGAVHTRGLDFACDFWVLVVVGGVLACYSLLVTVAFIIFWVRKRWQNEKLGLDAGDEYEDENLYEGLNLDDCSMYEDISRGLQGTYQDVGSLNIGDVQLEKP

SEQ ID NO: 9 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 10 - DNA, 2652 bp, Artificial Sequence
  Description of Artificial Sequence Synthetic polynucleotide
gctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccctgcagggagggcagaggaagtcttctaacatgcggtgacgtggaggagaatcccggccctactagtgccaccatgcagctgagaaacccagagttgcatctgggatgcgccctggcacttcgatttctcgccctcgtatcttgggacatccctggcgcgcgcgcactggacaacggactggctaggacccccaccatgggttggttacactgggaacggtttatgtgtaatctcgactgtcaggaagaaccggactcttgtatctcagaaaaattgtttatggagatggcagaactgatggtgtcagaggggtggaaagatgctgggtacgaatacctctgcatagatgactgttggatggcgccccagcgtgacagcgagggacgcctgcaggctgatcctcagagattcccacatgggattcgacagctcgctaattacgtgcactcaaaaggcctgaaactggggatctatgccgacgtcgggaacaaaacctgtgcaggattccctggaagtttcgggtactacgacatcgacgcccaaacctttgcagactggggcgttgatctgctgaagtttgacggatgttattgtgacagcctggagaaccttgccgacggctataagcatatgagcttggcattgaaccgcaccggacgaagcatagtgtactcttgcgagtggcctctctatatgtggccttttcagaaaccaaactacacggaaatacggcaatactgcaatcattggaggaacttcgccgatattgacgattcttggaaatccatcaaaagcatattagactggaccagtttcaatcaggaaagaatcgtggacgttgctggtccaggaggttggaacgatccagacatgctggtgattgggaacttcggcctcagttggaatcagcaggtcacacaaatggcgctgtgggctatcatggctgcaccgctgtttatgtcaaacgacctgcgacacatcagccctcaggctaaggcattgttgcaagataaggatgtaatagcaataaaccaggaccccctcggtaagcagggctatcagttacgccagggcgataactttgaagtgtgggaacgaccgttgtccggcctggcctgggctgtggccatgataaaccgacaagagattggagggcccaggtcttatactatcgctgtggcaagtcttggaaaaggagtggcttgcaacccagcatgcttcattactcagttgctcccagtcaaaagaaagctcggcttttacgagtggactagtcgacttcggagtcatattaatccaaccggcaccgtcttacttcagctggagaatacaatgcagatgtccctgaaggacctcctctagaggctaggtggaggctcagtgaaacttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcct

SEQ ID NO: 11 - DNA, 2721 bp, Artificial Sequence
  Description of Artificial Sequence Synthetic polynucleotide
gctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccctgcagggagggcagaggaagtcttctaacatgcggtgacgtggaggagaatcccggccctactagtgccaccatgtctaccgctgtgttggagaacccagggctcgggcgtaaactgtcagacttcggccaggaaacttcttatatagaagacaactgtaaccagaatggtgcaatctctctcattttcagtcttaaggaggaggtcggcgccttggctaaggttttacgtctgttcgaggagaatgatgtgaacctgacacatatcgagtccaggccatcccgcttgaagaaagatgagtatgaattctttacacatctcgacaaacggagtctccctgctctgacaaatataattaaaattctgaggcatgacatcggagccacagtgcatgaactgagccgtgataaaaagaaggataccgtcccatggtttcctcggacgattcaggaacttgaccgattcgctaaccagatattgtcctacggtgctgagctggacgcggatcatcccggattcaaggaccctgtatacagagctcgccggaagcagttcgcagacattgcctataattatagacacggtcagccgattcctagagtcgaatatatggaggaggaaaagaaaacgtgggggacagtgtttaaaacattaaagagtctctacaagacacatgcttgttatgagtataatcacatcttccccctgttggagaaatactgcgggtttcacgaggataacataccccagctggaagacgttagtcagttccttcagacttgcaccgggtttaggctgcgcccagttgccggcctcctcagttcacgggattttctcggcggcctggctttccgcgtgttccattgtacacagtacattcgacatggatctaaaccaatgtatactcccgaaccagatatttgccatgagctccttgggcacgtgccacttttctctgacaggagtttcgctcaatttagccaggagattggactcgccagcctgggtgcccctgacgaatacattgaaaaactcgcaaccatttactggttcaccgtggagtttggactctgtaaacaaggcgacagcataaaggcttacggagccggtctcttgagctcattcggggagctccagtactgcctgtctgagaagcccaagctgcttccccttgaactcgaaaaaacagctatccaaaactatacagtaactgagttccagcccttatattacgtggcagagagcttcaacgacgccaaggagaaagtccgcaactttgccgcaaccattcctcggcctttttccgtgcgttacgatccatacactcagaggatcgaagtgctcgataacactcaacagctcaagattttggcagacagcattaactcagagattggcatactttgtagcgcccttcagaagattaaatagaggctaggtggaggctcagtgaaacttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcct

SEQ ID NO: 12 - DNA, 3066 bp, Artificial Sequence
  Description of Artificial Sequence Synthetic polynucleotide
gctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccctgcagggagggcagaggaagtcttctaacatgcggtgacgtggaggagaatcccggccctactagtgccaccatgaaaacgctgagccaggctcagagtaaaacctcttctcaacagttcagtttcacaggcaactccagcgctaacgtcataatcggtaaccagaaactgactattaatgacgtggcccgagtcgctcggaacgggacactcgtcagcctgacaaataatacagatatcctgcaaggcattcaggccagttgcgactacataaacaacgcggttgagtcaggcgagcctatttacggggttacgtccggtttcggaggaatggccaacgtggccatcagcagggagcaggcttctgaattgcagactaatctcgtctggttcttgaaaaccggggctgggaataagctcccactcgcagatgtcagggccgctatgctgttgcgggctaattcacacatgcgtggggcaagcggcatccggcttgaactgattaagcgaatggagatttttttgaatgccggggtgacaccctacgtgtacgagttcggcagcattggcgctagcggggatcttgtccccttatcatatattacaggcagcctgattggactggacccctcttttaaggttgattttaatggcaaggaaatggacgcccccacagccttgcgacagctgaacctgtctcctcttactctgcttcctaaagaaggcttggctatgatgaatgggacatccgtgatgactggcattgcagctaactgtgtttatgatactcaaattctgacagcaatagctatgggcgtccatgccctcgacattcaggccttgaacggaacgaatcaaagtttccatccattcattcataacagcaagcctcaccccgggcaactgtgggcagccgatcagatgatctcacttctggcaaatagccagctcgttcgagacgagctggacggcaagcacgattatagagaccacgaactgatccaggatagatactccctgagatgtctcccacaatatctcggcccaatcgtggatggaataagtcagatcgcaaaacaaatcgaaatagagattaacagcgtgactgacaacccactcatcgatgtggacaatcaagctagttaccatggcggaaactttcttggacagtacgtaggcatgggcatggaccacctgcgatattacatcggactcctcgccaagcatctcgacgtccagatagcacttctggccagtcctgaattttctaatggattgcctccaagcttgttgggaaaccgagaaagaaaggtgaacatgggactgaaaggcctgcagatctgcggcaattccattatgccactcctgaccttttacggaaacagtattgctgacaggtttcctacccacgctgagcaatttaaccagaatatcaattctcaggggtacacctccgccactctggcacgacgatccgttgacatcttccaaaattacgtcgctattgcgttgatgtttggagtgcaagcggtagacctccggacttataagaaaactgggcattatgatgccagagccagcctcagccccgccaccgaacgtttatacagtgcagtgagacatgtggtgggccagaagcccacaagcgaccggccgtatatttggaatgataatgaacagggtctggacgaacacatcgctcgcatttcagctgatattgctgccggtggagtgattgtccaagctgtgcaggatatacttcccagtcttcactagaggctaggtggaggctcagtgaaacttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcct

SEQ ID NO: 13 - DNA, 4221 bp, Artificial Sequence
  Description of Artificial Sequence Synthetic polynucleotide
gctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccctgcagggagggcagaggaagtcttctaacatgcggtgacgtggaggagaatcccggccctactagtgccaccatgggagtgcggcatcctccttgcagccatagacttctcgccgtttgcgctctcgtttcccttgctacggctgcactcttgggacacatactgcttcatgatttcctccttgtgccaagagaactctctggcagctccccagtgctggaagagactcatcccgcacaccaacagggtgcctccagacccggcccgcgcgatgcccaggctcaccctggcagacctagagcagttccgactcagtgtgacgtcccacctaatagccgcttcgattgcgcaccggacaaggccatcactcaggagcagtgtgaggcaaggggatgctgttacatccccgctaaacagggtcttcagggagcgcagatgggtcagccttggtgcttctttcccccaagttatccgtcatacaagttggagaatctctcaagctccgaaatgggatacaccgccacacttactaggacaacgccgaccttctttcccaaggacatcctgacgctccggctggacgtgatgatggagacagagaacagattgcactttactatcaaggaccccgccaaccggcgctacgaagtgcctctggaaacaccacacgtgcattctcgagccccttctccactgtatagtgttgaattctctgaggagccattcggcgtgatcgtacgtagacagcttgatggtcgcgtcttgctgaataccacggttgctccattgttcttcgcggatcagttcttgcagctgtcaactagtctgccctctcagtacattaccgggttggccgagcacctgtccccactcatgctgtctacatcctggacgaggataactttgtggaaccgcgacctggctccgaccccaggtgctaatctgtacggaagccacccattttacctggcccttgaggacggtggaagtgctcatggggtgtttctgttgaactctaacgcaatggatgtcgtactgcagccgagtccggctctgagttggcggtccaccggtggcatcttagacgtgtacattttcctcgggcccgagcccaaatccgtggtacagcagtacctggacgttgttggctacccctttatgccgccttactggggcctcgggtttcatttgtgtcggtggggatacagctctactgcaatcactagacaggtagttgaaaatatgacacgggcacactttcccttggacgtgcagtggaatgacttggactacatggattcacgccgggacttcacattcaacaaagacgggtttagagactttcccgcgatggtccaggagctgcatcaaggaggaaggcgctacatgatgatagtcgaccctgccattagcagctcaggccctgctgggagctatcgcccgtatgacgaagggttgcgaagaggcgtattcataacaaatgaaacagggcagcctctcataggtaaggtttggcccgggagcaccgcctttccagacttcacaaatccaacagctcttgcatggtgggaggacatggtcgctgagttccatgatcaggtgccattcgatggaatgtggatcgatatgaacgaacccagcaacttcatacgcggtagcgaagacggatgtcccaacaacgaactggagaacccaccatatgtgcctggagttgtgggtgggacactccaggcggctactatctgcgcctcttcccaccagttcttaagcacacattacaatctgcataatctgtacgggctcacagaggccatcgcgtctcatagggcactggtgaaggcccgcggaacccgacccttcgtgatcagccgcagtacttttgcagggcatggtcggtacgcagggcattggactggggacgtatggtcatcttgggagcagctggcttcaagcgtccctgaaattcttcagtttaatttactgggggtgccacttgtgggggcggacgtgtgcggcttcctcgggaatacaagcgaagaactctgtgtgcgctggacacagctgggcgctttctacccgttcatgcgtaatcataattccttgctgtctctgccccaggagccttacagcttttctgaaccagcccagcaggccatgcgcaaggctctgactctgcgctacgctctgctcccccatctctatacactctttcaccaggctcatgtagcaggggagacggttgcgcgacccctgtttttagagttccccaaggattcttcaacctggaccgtagaccatcagctgttgtggggcgaggccctgctgattactccggtattgcaagccgggaaggctgaggtgaccggttactttccgttaggcacctggtatgatctgcagacagtgcctgtggaggctttgggatcccttcccccaccaccggccgctccacgagagccagctattcacagcgaagggcagtgggtcaccttacccgccccactggacaccataaacgtccatctgcgtgccggatatatcatccctctccagggcccaggccttactacgacagagagccggcagcagcccatggccttggctgtggctctgacaaagggtggtgaagccaggggcgagctgttttgggatgacggagaaagtctggaggtgctggagaggggcgcttacacacaggtaatttttctcgcccgtaacaatacgatcgtcaacgagcttgtccgtgttaccagcgagggggctggcctgcaactccagaaagtcaccgttctgggtgtagcaaccgccccccaacaggtgctgtccaatggtgtaccagtcagtaatttcacctattcccctgacacgaaagtgcttgatatctgtgtctctttactgatgggtgagcaattcctggtgagctggtgctagaggctaggtggaggctcagtgaaacttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcct

SEQ ID NO: 14 - DNA, 5736 bp, Artificial Sequence
  Description of Artificial Sequence Synthetic polynucleotide
gctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccctgcagggagggcagaggaagtcttctaacatgcggtgacgtggaggagaatcccggccctactagtgccaccatgcagattgagctctcaacttgcttctttttgtgtctcctgagattttgctttagcgccaccagacgttactacctcggggccgtcgaactgagttgggactacatgcagtcagacctgggtgagctgccagttgatgcacggttcccaccccgagtccccaagagcttccccttcaacacatccgtcgtgtataagaagactctgttcgtggagttcaccgaccacctgtttaatatcgctaaacctcgtccaccctggatgggcctgcttggccccaccatacaggccgaagtctacgacactgttgttatcacactgaagaacatggcgagccacccagtatccctccatgctgtaggagtgagctactggaaagcgtcagaaggcgccgagtatgatgaccaaacctcccagagggaaaaggaagacgataaggtgttccctggcggatcacacacttatgtctggcaggtcttgaaggaaaatggaccaatggcaagcgacccactctgtctcacctactcttacctcagtcatgtagatctggtgaaagatcttaactctgggctgattggagctctcctcgtgtgcagggagggctcactggccaaagaaaagactcaaaccctgcacaagttcattcttctgtttgccgttttcgacgaggggaaaagttggcactcagagactaagaattctctgatgcaggatcgagacgctgctagtgcccgggcatggcccaaaatgcatacagtgaacggttacgtgaaccgctcattgcctggactgattgggtgtcatagaaagtccgtttattggcacgtcattgggatggggaccacacccgaagttcactcaatttttctggaaggccatactttccttgtgaggaatcaccggcaagcaagtctcgagataagtccaattacattcctgaccgcccaaacgctgcttatggacttgggtcagttcctcctgttctgccatatatccagccaccagcatgacggcatggaggcctacgtgaaagttgattcatgtcccgaggagccacagttacgtatgaaaaacaatgaggaagccgaggactatgatgacgatctcactgactcagagatggatgttgtgcgcttcgatgatgataactctccttcctttatacagatccgcagcgtggcaaagaaacatcctaagacttgggtccattacattgctgccgaagaagaggattgggactatgccccattagtgttagcaccagatgacagaagctacaagtcccagtatctcaacaacggacctcagagaatcggtagaaaatacaaaaaggtccgcttcatggcttacacagatgagactttcaaaactcgtgaagcgatacagcacgaatcaggaatcctcgggcctctgctttatggcgaggtcggcgacactctgctgattatttttaaaaatcaggcaagccgtccgtacaacatatatccacacgggatcacagatgtacggccactctattcccgccgcttgcccaaaggtgtgaaacatttaaaggattttcctatactccctggagaaatattcaagtacaaatggacagtgactgtcgaggatgggcccacgaagtccgaccctcggtgtctgactcgctactattcatcctttgtgaatatggagcgcgacttagcttccgggctcattggacccctgcttatctgctataaagaaagtgtcgaccaaagaggcaaccagatcatgtccgataaaaggaatgtgattcttttcagtgtgtttgacgaaaaccggagttggtatcttaccgagaacattcaacgcttcttacctaacccagctggcgtgcaactcgaagaccctgaattccaagcgtccaatataatgcactcaatcaatggctacgtgtttgacagcctgcaattgagtgtgtgcctccacgaggtggcttactggtacatcctgtctatcggcgcccagaccgattttctttccgtcttcttcagcggttacacctttaagcataaaatggtgtacgaggacactcttacactgttcccgttttctggcgagaccgtgtttatgtcaatggaaaatcccggtctgtggatcctggggtgccataattctgatttcagaaaccggggtatgacagcactgctgaaggtttccagctgtgacaaaaacaccggcgactattacgaggattcctacgaggatatttccgcctacctcctgagcaaaaacaacgccattgagccccggagttttagccagaaccctccagtccttaagcggcaccagcgcgaaataactcggacgaccttacagtccgatcaggaagagattgactatgatgacacaatctccgttgagatgaaaaaagaggactttgatatttatgatgaggacgaaaaccaatcccccaggagcttccagaaaaagacgcggcattattttatcgccgccgtggaaagactctgggactatggaatgagctccagcccccacgtgttgcgaaaccgcgctcagtctgggtccgtcccacagtttaagaaggtcgtctttcaggaatttacagatgggtcttttactcaacctctttaccgaggcgagctgaatgagcatctcggcctgctgggaccctatattagggctgaggtggaagacaacattatggtcactttccgcaaccaggcatcaagaccatattccttctattcctcccttatcagttatgaggaggaccagcgtcaaggcgcggaacctaggaaaaatttcgttaaacctaacgagaccaaaacctacttctggaaggtccaacatcacatggcgcccacaaaggacgagttcgattgcaaagcgtgggcttacttcagcgacgtagacctcgaaaaggacgtgcatagtggactgatcggaccgctgctggtgtgccacacgaacacccttaatccagcccacggccgccaagtcacagtccaagagtttgccctcttcttcacgatcttcgacgaaaccaagagctggtattttacagagaacatggagaggaactgtcgtgcgccatgtaatatccaaatggaggaccccaccttcaaagagaactatcgatttcacgctatcaacgggtatattatggacacacttcccggcttggtgatggctcaggaccagaggattcggtggtaccttctgtctatggggagcaatgaaaatatccattccatccatttttctggccacgtttttactgtcagaaaaaaagaggagtacaaaatggctctgtacaatctttaccctggggtatttgaaactgtcgaaatgttgccatctaaagcgggaatatggcgcgtcgagtgtctcattggagagcatttgcacgcaggaatgagcaccctgttcctcgtgtacagtaacaaatgtcagacccctcttggtatggcttcaggccatatccgggactttcagattacagcatctggccagtatggacaatgggcgcccaagctggcccgactgcattacagcggctctatcaacgcttggagtacgaaggaacccttttcctggataaaggtcgatctgctggcccctatgataatccacggaatcaagacccagggggcccgccaaaagttctcaagcctgtatatttcacaattcattatcatgtacagcttggatgggaagaagtggcagacttatcgggggaacagtaccggcaccctgatggtcttttttggaaacgttgacagttcaggcattaaacataatatctttaaccccccgattatcgcccggtatatccgcctgcaccccacacattacagtatacgtagcactttgagaatggagctgatgggatgtgatctcaacagctgttccatgccattgggcatggaaagcaaggccatcagtgatgcacagataactgcttcaagttatttcaccaatatgtttgcgacctggtcacctagtaaagcacgcctgcatcttcaaggccggagtaatgcctggcgaccccaggtgaacaaccccaaggagtggttacaggtcgatttccagaaaaccgtgaaggtcacaggcgttacaacccagggcgtgaaaagcctcctcacctctatgtacgtcaaggaattcttgatttcctcaagtcaggacggacatcagtggaccctgttctttcagaacggaaaagtcaaggtgtttcaggggaaccaggactcttttacgcccgtggtcaattctcttgatcctcccctccttaccagatatctgaggattcacccccagtcatgggtccatcagatcgcgctcagaatggaggtgctggggtgcgaggcacaggatttgtactagaggctaggtggaggctcagtgaaacttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcct

SEQ ID NO: 15 - DNA, 8418 bp, Artificial Sequence
  Description of Artificial Sequence Synthetic polynucleotide
gctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccctgcagggagggcagaggaagtcttctaacatgcggtgacgtggaggagaatcccggccctactagtgccaccatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgaaggctaggtggaggctcagtgaaacttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcct

SEQ ID NO: 16 - DNA, 2610 bp, Artificial Sequence
  Description of Artificial Sequence Synthetic polynucleotide
gctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccctgcagggagggcagaggaagtcttctaacatgcggtgacgtggaggagaatcccggccctactagtgccaccatggaaaccgatacactgctgctgtgggtgctgctgctgtgggtgccaggatctaccggtcagtctgtgttgacgcagccgccctcagtgtctgcggccccaggacagagggtcaccatctcctgctctggaaccaggtccaacattgggagtgattatgtttcctggtaccaacacctcccaggaacagcccccaaactcctcgtttatggcgataatctgcgaccctcagggattcctgaccgattctctgcctccaagtctggcacgtcagccaccctgggcatcaccggactccagactggggacgaggccgattattactgcggcacatgggattacaccctgaatggtgtggtgttcggcggagggaccaagctgaccgtcctaggttctagaggtggtggtggtagcggcggcggcggctctggtggtggtggatccctcgagatggcccaggtgcagctggtggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgcgcacttcttacctgaaccatggtgattactggggtcaaggtactctggtgaccgtgtctagcgccgctgcattcgtgcctgtgttcctcccagctaagcccactaccacccccgctccaaggccgcccacgcccgctcctactattgctagtcagcctttaagtttacgacccgaagcttgcaggcccgccgccggcggcgctgtgcacaccagggggcttgattttgcctgcgacttttgggtattggtagtggtgggcggagttttagcctgctacagcctcctggtaacagtggcttttatcatcttttgggtgaggaaacgatggcagaacgagaagctcgggttggatgccggggatgaatatgaagatgaaaacctttatgaaggcctgaacctggacgactgctccatgtatgaggacatctcccggggcctccagggcacctaccaggatgtgggcagcctcaacataggagatgtccagctggagaagccgtgactcgagaagcttaagtttaaacttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcct

SEQ ID NO: 17 - DNA, 1899 bp, Artificial Sequence
  Description of Artificial Sequence Synthetic polynucleotide
gctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccctgcagggagggcagaggaagtcttctaacatgcggtgacgtggaggagaatcccggccctactagtgccaccatgcactcttcagctcttttatgttgtctcgttttgctcacaggcgtccgcgcctctccgggtcagggtacccagtctgaaaactcttgcacccattttcccggaaatttacccaacatgctgcgcgatttacgagacgcctttagccgcgtaaagaccttctttcagatgaaggaccaactggacaacttgctgctgaaggaatctttgcttgaggattttaaggggtatctcggctgccaggccctgtctgaaatgatccagttctacctcgaggaggtcatgccacaggcagagaatcaggatccagacattaaagcacacgtgaattctctgggcgagaatttgaaaaccctcaggctgcgcctgcggcgatgccacagatttcttccttgcgaaaacaagtcaaaagctgtcgagcaggtgaaaaatgccttcaacaaactccaagaaaaggggatatacaaagccatgagcgagttcgatatattcattaattacattgaggcatacatgacaatgaaaatcagaaactaaaggctaggtggaggctcagtgaaacttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcct

SEQ ID NO: 18 - protein, 1368 aa, Artificial Sequence [sequence omitted]

SEQ ID NO: 19 - RNA, 100 nt, Homo sapiens
  misc_feature (1)..(4): modified with 2′-O-methyl phosphorothioate
  misc_feature (97)..(100): modified with 2′-O-methyl phosphorothioate
cgugaguaaaccugaaucuuguuuuagagcuagaaauagcaaguuaaaauaaggcuaguccguuaucaacuugaaaaaguggcaccgagucggugcuuuu

SEQ ID NO: 20 - DNA, 598 bp, Artificial Sequence
  Description of Artificial Sequence Synthetic polynucleotide
gctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactc

SEQ ID NO: 21 - DNA, 665 bp, Artificial Sequence
  Description of Artificial Sequence Synthetic polynucleotide
ttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcct

SEQ ID NO: 22 - DNA, 360 bp, Homo sapiens
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaa

SEQ ID NO: 23 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 24 - DNA, 23 bp, Artificial Sequence
  Description of Artificial Sequence Synthetic oligonucleotide
cgtgagtaaacctgaatctttgg

SEQ ID NO: 25 - DNA, 2079 bp, Artificial Sequence
  Description of Artificial Sequence Synthetic polynucleotide
gctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccctgcagggagggcagaggaagtcttctaacatgcggtgacgtggaggagaatcccggccctactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactcgagaagcttaagtttaaacttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcct

SEQ ID NO: 26 - protein, 288 aa, Artificial Sequence
  Description of Artificial Sequence Synthetic polypeptide
MSRSVALAVLALLSLSGLEAIQRTPCREGRGSLLTCGDVEENPGPTSATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 27 - DNA, 20 bp, Artificial Sequence
  Description of Artificial Sequence Synthetic oligonucleotide
tgagagggcatcagaagtcc

SEQ ID NO: 28 - DNA, 20 bp, Artificial Sequence
  Description of Artificial Sequence Synthetic oligonucleotide
aagtcacatggttcacacgg

SEQ ID NO: 29 - DNA, 21 bp, Artificial Sequence
  Description of Artificial Sequence Synthetic oligonucleotide
gctatgtcccaggcactctac

SEQ ID NO: 30 - DNA, 20 bp, Artificial Sequence
  Description of Artificial Sequence Synthetic oligonucleotide
aggatgctaggacagcagga

SEQ ID NO: 31 - DNA, 3677 bp, Artificial Sequence
  Description of Artificial Sequence Synthetic polynucleotide
tgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccctgcaggtgaaagaccccacctgtaggtttggcaagatagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgattgactgagtcgccctgatcagtactactagtgccaccatgcagctgagaaacccagagttgcatctgggatgcgccctggcacttcgatttctcgccctcgtatcttgggacatccctggcgcgcgcgcactggacaacggactggctaggacccccaccatgggttggttacactgggaacggtttatgtgtaatctcgactgtcaggaagaaccggactcttgtatctcagaaaaattgtttatggagatggcagaactgatggtgtcagaggggtggaaagatgctgggtacgaatacctctgcatagatgactgttggatggcgccccagcgtgacagcgagggacgcctgcaggctgatcctcagagattcccacatgggattcgacagctcgctaattacgtgcactcaaaaggcctgaaactggggatctatgccgacgtcgggaacaaaacctgtgcaggattccctggaagtttcgggtactacgacatcgacgcccaaacctttgcagactggggcgttgatctgctgaagtttgacggatgttattgtgacagcctggagaaccttgccgacggctataagcatatgagcttggcattgaaccgcaccggacgaagcatagtgtactcttgcgagtggcctctctatatgtggccttttcagaaaccaaactacacggaaatacggcaatactgcaatcattggaggaacttcgccgatattgacgattcttggaaatccatcaaaagcatattagactggaccagtttcaatcaggaaagaatcgtggacgttgctggtccaggaggttggaacgatccagacatgctggtgattgggaacttcggcctcagttggaatcagcaggtcacacaaatggcgctgtgggctatcatggctgcaccgctgtttatgtcaaacgacctgcgacacatcagccctcaggctaaggcattgttgcaagataaggatgtaatagcaataaaccaggaccccctcggtaagcagggctatcagttacgccagggcgataactttgaagtgtgggaacgaccgttgtccggcctggcctgggctgtggccatgataaaccgacaagagattggagggcccaggtcttatactatcgctgtggcaagtcttggaaaaggagtggcttgcaacccagcatgcttcattactcagttgctcccagtcaaaagaaagctcggcttttacgagtggactagtcgacttcggagtcatattaatccaaccggcaccgtcttacttcagctggagaatacaatgcagatgtccctgaaggacctcctctagaggctaggtggaggctcagtggtttaaaccgctgatcactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcccgggcacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctggccggcc

SEQ ID NO: 32 - DNA, 20 bp, Unknown
  Description of Unknown Target sequence
agagcaacagtgctgtggcc

SEQ ID NO: 33 - RNA, 20 nt, Artificial Sequence
  Description of Artificial Sequence Synthetic oligonucleotide
agagcaacagugcuguggcc

SEQ ID NO: 34 - DNA, 20 bp, Artificial Sequence
  Description of Artificial Sequence Synthetic oligonucleotide
cgtgagtaaacctgaatctt

SEQ ID NO: 35 - DNA, 75 bp, Homo sapiens
ctggatgacgtgagtaaacctgaatctttggagtacctgaggaatatcgggaaaagacacattaatattgccagg

SEQ ID NO: 36 - DNA, 58 bp, Artificial Sequence
  Description of Artificial Sequence Synthetic oligonucleotide
ctggatgacgtgagtaaacctgaggaatatcgggaaaagacacattaatattgccagg

SEQ ID NO: 37 - DNA, 72 bp, Artificial Sequence
  Description of Artificial Sequence Synthetic oligonucleotide
ctggatgacgtgagtaaacctgaattggagtacctgaggaatatcgggaaaagacacattaatattgccagg

SEQ ID NO: 38 - DNA, 73 bp, Artificial Sequence
  Description of Artificial Sequence Synthetic oligonucleotide
ctggatgacgtgagtaaacctgaatttggagtacctgaggaatatcgggaaaagacacattaatattgccagg

SEQ ID NO: 39 - DNA, 74 bp, Artificial Sequence
  Description of Artificial Sequence Synthetic oligonucleotide
ctggatgacgtgagtaaacctgaattttggagtacctgaggaatatcgggaaaagacacattaatattgccagg

SEQ ID NO: 40 - DNA, 69 bp, Artificial Sequence
  Description of Artificial Sequence Synthetic oligonucleotide
ctggatgacgtgagtaaacctgaggagtacctgaggaatatcgggaaaagacacattaatattgccagg

SEQ ID NO: 41 - DNA, 56 bp, Artificial Sequence
  Description of Artificial Sequence Synthetic oligonucleotide
ctggatgacgtgagtaaacctggaatatcgggaaaagacacattaatattgccagg

SEQ ID NO: 42 - DNA, 69 bp, Artificial Sequence
  Description of Artificial Sequence Synthetic oligonucleotide
ctggatgacgtgagtaaacctgtggagtacctgaggaatatcgggaaaagacacattaatattgccagg

SEQ ID NO: 43 - DNA, 75 bp, Artificial Sequence
  Description of Artificial Sequence Synthetic oligonucleotide
  modified_base (26)..(26): a, c, t, g, unknown or other
ctggatgacgtgagtaaacctgaatnctttggagtacctgaggaatatcgggaaaagacacattaatattgccag

SEQ ID NO: 44 - DNA, 66 bp, Artificial Sequence
  Description of Artificial Sequence Synthetic oligonucleotide
ctggatgacgtgagtaaacctgagtacctgaggaatatcgggaaaagacacattaatattgccagg

SEQ ID NO: 45 - DNA, 73 bp, Artificial Sequence
  Description of Artificial Sequence Synthetic oligonucleotide
ctggatgacgtgagtaaacctgactttggagtacctgaggaatatcgggaaaagacacattaatattgccagg

SEQ ID NO: 46 - DNA, 71 bp, Artificial Sequence
  Description of Artificial Sequence Synthetic oligonucleotide
ctggatgacgtgagtaaacctgaatggagtacctgaggaatatcgggaaaagacacattaatattgccagg

SEQ ID NO: 47 - DNA, 71 bp, Artificial Sequence
  Description of Artificial Sequence Synthetic oligonucleotide
ctggatgacgtgagtaaacctgtttggagtacctgaggaatatcgggaaaagacacattaatattgccagg

SEQ ID NO: 48 - DNA, 68 bp, Artificial Sequence
  Description of Artificial Sequence Synthetic oligonucleotide
ctggatgacgtgagtaaacctgagagtacctgaggaatatcgggaaaagacacattaatattgccagg

SEQ ID NO: 49 - DNA, 70 bp, Artificial Sequence
  Description of Artificial Sequence Synthetic oligonucleotide
ctggatgacgtgagtaaacctgaatgagtacctgaggaatatcgggaaaagacacattaatattgccagg

SEQ ID NO: 50 - DNA, 67 bp, Artificial Sequence
  Description of Artificial Sequence Synthetic oligonucleotide
ctggatgacgtgagtaaacctgaattacctgaggaatatcgggaaaagacacattaatattgccagg